SEQUENCE LISTING (21 sequences)

SEQ ID NO: 1 - DNA, 7001 bp, Homo sapiens
  misc_feature: Human FABP-3 Promoter and coding sequence
aagattggtagaatgaatgaatatttaaagagagagaaagggaaatatgaggaaacttatgtccaagatctgaaggccactctgtgacctcaatgaagaggagttggccatatacttgaatcctttcctacttattcagggtctcaaagggccttggcttactcaaatatcccggggaaaagaactggaaggaggaaaggagaaaggagttactattccttcaatattagtgcacggtaggctttccatttgttatctcttttaattgtcctaacaaccctgagaagttgatggccttcctctatttgggagaaaacaagactcagaagggggaaagggagcttgttctaggtctctagctagtaaatgcaggcctggagtttagaatcagggctctttccaaagacactgcatctgaagatgtcagaggcattctaaccagagtgactccatcttgaatagaggctggataaaatgaagctgagacctggctgggatgcattcccacaaggtgaaacattcttagtcacaaaatgtttacagttaagagttaagggaacaggttaataatttactaaacagacatttctttacttctctaataaacttgcttttactttattctgcggatttgccccgaattctttcttgtgtgagatgcaagaactctctcttggggtctggattgggacccctttctggtaacaaagaccccatggaccaagggcataaaaagaagggatgtctaagacttgctggttctatggcaggcagaggcagaaagagccccaccatcattcccagcaaatgggaaaagttcccagttgcagatattaggggtgggatggggcgggggtagtcagcaaccatagacttagaccctgaagaggcaaaaaaggagggccatgttcttgggtcagcagagcttctactcagcttcttcagcctctagctctttcctggtgctagtagcacattctctagtggaggcatccagatggcagggagggtccaggaaacagctgaacatgctgagcaggcctcccttgtccccgctccccatggccccatggatcatccggtgctgcagctcatctcattggctggcttctggttactcatctctcctcttctccatcttcccagcctgtggttgccgtggaaacatagaacagtgacctcaccataggatgagggctggggagatgctgttcttggcaggcgctggtcttgctgcctgccagatgttatatccacatctggatctgccaagggcaagacagaggagtacaaaaagggacagggctcacattcttccccaaatgtagtcagcataacagaggaagaggatgcaggtagatcagagggatgaggagagaactgtacatacttagaagggagaagggtgtaaaccaggaggaaaggagatggggtgtggcttatgggttaacctcggtgacccaggtctgagcaccatggcgaggtgagggaagggcagtgagggtagttggtctgaggtttgtcgagtagagctcctcaaagccccgtgctgctcaatttggacaggcccaagagagggagcaaatggattttctggcagtgaccccaccccaacccattctgaaaaacctctatagcccctttttcagttgaagccctttctaatcctgtcccagaaataataaaactgaggattaaagaagcaaagcaatctgcctcaggtaactaactcagtaggtagtggcagagctcagactaggtcttaggtgtctgcactatccctggagacaataacaatagcagcaggaggagcagcagcagtgaactttcgtaagtcaaccatgcttagcatttaaatgtattatctcgggccaggtgtcgtggctcacacctgtagtctcagcactttggaagccgaggcaagcagattgcttgaggccaggagtccaacaccagcctgggcaacacggcaaaaccctgtctctaccaaaaaaatgcacaaaaaattagccaggcgtggtgccatgcgcctgtagtcctaactactcaggaggctgaggtgggaggatcccttgagtctgggaggtcaagaatgccatgagctgagatcatgccactgcactccagcctgagtgacagagtgagactctctccaaataaataaataggccaggcgtggtggctcatgcctgtaatcccagcactttgggaggccaaggcaggcggatcacttgaggtcaggagttcaaggccagcctggccaatatggtgaaaccccgtctctactaaaaatataaaaattagcctggcgcggtggcccgcgcctatagtcccagctacttgggaggctgaggcaggagaatcacttgaacctgggaggctgaggcaggagaatcacttgaacctgggaggcagaggttgcagtgagctgagataacaccattgcactccagcctgggtgacagagtgcaagactccatctccaaaaaaaaatcccttcattttaaagatgaaactgaagctggcgtggtggctcatgcctgtaatcccagaactttaggaggtccaggtgggcagatcacctgaggtcaggagtttaagaccagcctggccaacatggcgaaaccccgtctctactaaaaatacaaaaattatccagatgtggtggcagttgcctgtaatcccagctacttgggaggctgaggcaggagaattgcttgaacccaggaggtggaggttgcagtgagccaagatcacaccactgcacttcagcctgggcaacgagactccttctcaaaaaaaaaaaaaaaaaaatggagactcagaatggcttaagtgaattgcccaaggccacacagctagtacccaacaaaattcagatcagaacgcagatctgccagcagccattagcccagccccagctctccatcattgcattacaagggccacataaggagtcagttctagttcctggccacacagtccttgagaggaggccctatgtgtgagggctgcgaccttgagtcagaaatcatcttttccccctcctcctctcctggcacctgttccaacagtgagatcaccaaggctccatgcccagccctgaagcctgtccctatagctttggcagaaatgagcctctgagtcttctggcaactggaccgtccaaaggaatcaagatcttagggtggggagagggctcttcctccctggccttgttctcagaacagtccagggaagggaggcagggtttgcacatggagaaacagagtcccaggaaggagggcctgccatactctacgcacgtggcaggcctctgggctcctgtgttggctcctgcaggcccaccctggacaggctgctgctggctgaaattcagttcggcactactccttggtgtgcagggcctcctgccatccctcagaaattcatttatacctcacttcctttgagaagtctacattgggccctcaggtcaggacaggacaggactccctttagaggcaccacgctagttttatgtctggctgcccagaggtgaatgtgtgtcttgttccctgttgtctctctccaaagcagggcctggcacaaagtaggtgctttattaactgctgaattaatgagtgagctcctcagagcttcaacttgttcacctttttaaaaagagaaaataatacagtaacacctacttcatggattttgctttaaaagtccaatgaggccaggtgtggtggcgtgtgcctatagtcccagctgctggggaggctaaggtaggaggatcccctgagccccggagtttgaggctgcagtgagctatgatggcgtcacagtactccagcctgggagacacagcgagagactttgtctctaaaaaataataataaaataaaaagttcaatgaaacaatacacccaaagccctcagcatgcaataaatagcaagacaaggcaggtcttatttttactgaaagtgcttagtaaactatacagtgacaaaccaccgcacaacaggctctcgaaaggaggcagcaaattacccaaaagtgcaggcggcttgctagtgtgcacaggccaaagaaagggcggcaggtggggaaggcagccatgggccttgaagagctgaccgaattggcagaatttctgcaggaggggagctgggaacgacctgagctaaagctcggagctgtgcgaagaaaccggaaaagcccagagcacttgcaggggcgggtggggagctagatggtggggtggggtggggacggaggagggccagcaggagacattccgcagggaggggcaagcacgtgtgaggcgggcggggcgcgaagggtcaggcttttgctcaaaacaggcagaggacaaggtcagctcagccgcagaccgagccgctggtgactgtctccgccaccaggcagtgagagtgaagggagagcgcgagctctgaagcccgctagactaagcttgcaatctgagctccattcaccccctcctatttcttgagaccttgtcagttcccctgtgagcctcggactcctcacttgtaaaacgaggacagatgcccgtgccagaagtcaaccagagctttccccggcgtgggcaccagcccaagggcgttttgcttttctagtctcatctctgctctgacgctaagctcaaagagggactgggggacgggaagatatccaccatggcatgcgccctagctctcgggctggtgttggctgcttccttctcagattccagagtgcctagaggccaggaaagggagaaggtcctaccagcctggggtagggactcgggggccaggcactggcgctgacgcaggctagcagggcgccactggctggtccccacccacctcggtgggttgggggatgggcgcaccagcccctcctgggtgagccctagcctggggcttcctatttcgggagccgggggcgtgggccacgtctcctcatgtgatgcgagggctatttaaagcggcagcccgggcagggagccgccgtcggagcccttgcacgcctgctctcttgtagcttctctcagcctagcccagcatcactatggtggacgctttcctgggcacctggaagctagtggacagcaagaatttcgatgactacatgaagtcactcggtgagcaagccgcggggctcaggatgttggcttggggactggctggtggcgtgcctagccccacgcagcactcctgccgcatccctcctggttaagactggggaataggggagcgcggagatggcagcctggcctagagcaggtggggcctgttcagagggggctttggtggtccaaatctggttagagaccacggtagggaggtggtggaaggaggcagctgtgtgggaggctctttccaggaagagggatatgtgatttggaggtaggaggagggtttggataaagaacactgatcacaggaaagggagtgtagccaggggagaaaaagaacaggggcatgggtagtttagaaattggaggagactgaacccagaaagggaatggggcagccagggagtgtacaatgatgtaaacaagtaggaaatacctaggaggaaaaagattagtggggaaaaaactgtggatcagtgaatcagatatgagaaggacgtaagacaggaacctgcagtaagcagcaatccccatctctgcttggttagggaagagaattcttgctggagaatgccctttctcaccagccagtctgaccttgtcctgcagtctatgtatccaggccttcatcactgtctgtgagcctcgtggtagggtggggcaagaggcccatgatcagctgggcctttcctgcaacccaaggctcacctatctgtgcgaggggtaggcagagaaagccattggacttctgatgtgcagtagagggtcccaaggcaaggtcaagacctgggagggaggatcactggtttaggaggatgtggagaactgctgtggtgttgggatggagaagaatcaggattcaaagaatctcacaggtgaggaacttggagattcccataccatctagttcaacagggaaactgaaaccaggagagtagaaatgtattataacaattccacagcagagccaatatgaaaatctaaggtttctagatctgtaacccagagctcttcccactaccctacaggccctgcgagtgggaagaaaagtagaaactgcttagctaatgattgacctcagcccttcttctactgctttgggcttagatggagaggtcaaagctctcaacggcctctaccctatcttgggcgctatgcccagtaattctaggcaggcagtcattcttagaggagcagcccccagcccccacgaacacagcccagcagctattgggaagttggaatgcccagatttagttcctccttccaaagctgggccagagctgagtcttgaattgagctgcaacaactttaccattcttgttcccttattctgccccgagttgggtcagcgggctggtctccctgaagtcctgttatctttcagcagcttatgttaaggcagccagcattctcatcgtaggaatggaaagcctgggaaaataccctcctcagctctcagtaagtagtgctggcttcatttctaagtagaacccagatctccctgagtctcctaaattctgtcagctcaatattcttagtttctcttggttcagaccctcactcatcccgcagtggtttccttttcaaacactccatacctctgggtagatcctaagtgaacagagctcccagtgccgtgacaaggtcctgctctgtgcaagggagtgtgattggcctgactcatcctgataccaaggggcaatgccaagttcctcactggccaagcaagggtgggctgacagcataacagcagaggcagcccctgcccctcctgctgtagacctagggctctcaaggggcaaagaggtcccgtctagtaccagtgaccacaggcacaactgctggcctggattgagtatgtgctggaca

SEQ ID NO: 2 - DNA, 7001 bp, Homo sapiens
  misc_feature: Human FABP-5 promoter and coding sequence
gggatttcagacccacattcagaatcctgaggccatggtagttttgagatctcctctttgtagccctgagtgttgacaagcccagcttggtgttcaatatttggcaactcagtaggatgtctgagaccctgcagaattaactatgtatttcaaggttgtaagataggagacaagatttgagatttgtggcacaaatattttttatcccaccttccaaaataggctggaagtaagatcacagttgctcttcttaatggtaacaactacttagcctcccagttcccgcatgaaattacaatatttaaccaatgtagaaaacagaatatactttactggaaacttcccctacatactgttgaatctgtatctttaagtttggtcaatgcagaagtgagcaggtgaacaaaaagtgtcctgtggtgtctatgggtgggtgaacaaaaggtacccatcctgctgcttgggtccttcagagaagtgcacaaaatcagctgcatgctggttttacagctctgtgcatgactctgaggagttatagggtagctcttcccactgccaaagtaggaggaccagagataggatcaagaaagcatcttcagtcggatttaggtaggttattaagtgctgatgattcttcttcaaagctaatccagaaaaatggaagcagtgggcctgactcactgagataaatagcttagaggcaagcaaatccacggttttgtagatttgacttgaaaaaaatcattaattcttatagataaatatcaaaatagagaaatgatcaaatggtgtgagaatctaggaatactgcatgggaggtaggaatgaaaaatagaaaagggtaatctacaagaaaagaatttggcctcttccagtgggaacatatcatttggaggttttttatgtttgaaattttttaaggacagagatgctcattattaatgtgtctgtgtgtttgggcttggccagcctaagattttgtatataaggaggcttaggagtaataatgtgattagaagcgggcttgaagaggttggggttgctaaggaatttgttttgaagttgcattgcctggtaaacatagtgcttttactaagattttgtgtttattggggtggctttgctagcatcctcccgctccatttcaagctgtgccctttggagttgtgcttttaaacctttgaacactgctcatatgaggtataaaagggttatcccaaactatgtgtatctgttgccatgagctattgacaaatgtaatatacaataatacacatgtctggaaagtaggaagatctatataagtaagggaaatgtatgaattttatactgaaagcttttattataaaatgagccattatgtcctctgccttattttgaagccattcactttttattactgtaacttttgcaatgcctcattttccctccagctatcctctagtccagttaactcagcaagcacaagtttatgatggatcatttaaggagctggaaaattttaatggttagccgtggcagttatgatgtgtaagactctaactgctttaagtaaaaggacaaagttcaaaatataactccatctttataagtgctttaattaaaaccaatcttattatgaaaaacaaaccaaaaaaaccttgcattgatggatggtagctatttgcaatttcttgttttggctggatgcattgaaggattaaaaatttaatatttaaggtgtgccttaaactgcaaggttccctgattttattctcatctaggaatttttgctgctttaggtagctgacaacatgcagatccatactctatctcttaagattttcttttggaactgattccaggtgaaattttcttagggaagatgtggctagaagctgggtatgcaaagcatgtctatagcaaggaaagccagatgagtgagggtcaaaaatctagttataaaatttaagggagacactaggtttaaataaacatattctccaaccttagtgattaatttttttccttttttgatcatctggccatgtacttagagaagactcagttccctgaaaatggattactttgaatattctctgcaataatttaatgtagtactctccatttctctaccaactgcctgttgctaagccaccaacacttaactgtccttaggaagaactaatatacaataatcaactttgtggaaggcacatctatgtacataaactttaactgggaagttgtctttaaataaaaatgacacaatatgctccaaagctgtgtgtgattacagaatgagaagctgaaaaactactcacgtttatcaagtaaagcctcagagaaatattacggtttcaccactaggtggaaatagagtaattttgagtacgctttttcaaggccatgaacttctactgcaggagtacttcataaactgaaggggacctaaaaaatcatttagtattaattctcatctccttaaattcatagatgataaaactaagcttcagagatattaagtaaaattgcatctgtcttgaaaaaggcagttttctaagctactggaagaagagttgctagtaattcttgattcttcagtcattttcatccataagaataatgtgttattatcttccagtgtgaaggaaaacatgatataacacctgtattacctatgtgctgttactaaaaataatgaacagcacagttactaatgttcgtaaggacaggttaggataaaactattgtttcttgtttcctatgttgtaattaatcacaatcaagtgatgattctcctccttctttccaagaagaaatactcaaccatctccataaagttatatgagacttcagtgaaatttgatttcacaacatttttggttttacacctcataatgggtgcaaggtaagtagcctagaataggcctttacttagcgggtgaaggcagtacttaactgctttttcacagcttaaaatccattaccattactctggagaaggaaagaatctgagtagtagagcttaaggggcattagaaataatctactccaatcatacatctgtatatgcatcagatgtggtgtctgaaaacaagacattcaatgtgtttccctgaggaacagagcaatcaccatgcccttttttgttccacaagatacaataacggttattattctggccatatagctttgttttgtctgtatgttttaacacaaaagaattatttccactcatcacatcagagattgtgtttaaacttaagttttaagtaaggccttgggattaaaggctatttcttaagtacctaagtatctagtactccagtgccattatctttttgatatttctgtaacatgttagattatgatcatttaaaaattagtatcagagtgctgcgtcctattattactacatatagtctatacagaaatggataagacatgttgaaatagcacatgaagagctcaagttcaaacacacaggcagccctaaggagctgcctgaagtgcctggtagccagggcttctcatcactgagtaaatgatgcactcagcttccctcagaacacgaatattttctgagcagtagggcctcgggtgtgggacatgcagacccttattgggaaaacaattgagtgttaaatggtaccacaaggaagactggtgtgaccctaaactgagggcagggacttgaaatgaaagggatgcagttcaggggagtacatgtcctgcagcatgttgggagatagcaaaccaacctcagatttcaattttgtcaaggatgatacaagaactagtagtgtctcggtgactgtggtctactcagagctccatcacagctaccctgagcccaaggcagtcaggtgtggtctgatttcataaggtcttcagtgagacggacctggatggggattctggttctaccactgtgagaactatctttgccctattttctcttttgaaaaattgggataacacccctagcaaaaatatagtggagaccaatgtctataaagtgcccaggaagcacatctagcacttagtagctactcaataaatgcacattaccgctatcgaaacggccagcctgtgtggcaacttcctcacctcagtccatctgacttccaacaggcacccgggagcaccaagtaataaattgtgaagagtacactttgaaacgtatcatgttctacgcacatgtgtagggtattattatagttcaagagaaagagggaacccgctggtgtctgagaagaaacccgctggtgtctgagaagaaacggtgaagggtgcactacgaagggaatataaactaatcaaacattcttgaatggcaatgacagtcactaaagttgacacttcctgcagtcttttagtttagaaaccacacttcccgagacgtcccccatcccaacagggataaaatcctctagctcaactcaggtatatccccactattgggccagggagacctgcggatggcgagagcaggttctcttaggagggtgtggattgtgagcttgtgcgctggcgaggagcagaaggtcagggaggcaccgtagttaatgaatcaataaaccaaacagccgatcaatcacttcctgcagtccgagtccggcgagggctaatcgattggtccgggtattttgaaaggaaaaacggcggtggatatgttgtcaaacggaggggtgcaggagaggcggggccaggcctccaggtgacccctctggcttcctgggcctgcagaggcgccgaaacgtgcaggcgccggcgtccatgaggcgtggggcgcgccgggcgagtccctgcttgcaggaggcgcccgagaggccgtcgcgtaccctggcaagaggagctggttagcacctcccgaccccgagaaggcccagggggcgcccggcgggcggggcggggaccgggcgaggcccgcctcccattggccgcatagcgccgcccagcgcgggccgccgttataaagcagccgccggcgccgggtgcctcacagcacgctgccacgccgacgcagacccctctctgcacgccagcccgcccgcacccaccatggccacagttcagcagctggaaggaagatggcgcctggtggacagcaaaggctttgatgaatacatgaaggagctaggtgaggcacccggcctggcagcgcctgcaacgtggcgtgttgtgcggtcgtctgtccctaggtccccgtcagcgtgccagattctggggcaggagatgctcggcggcctacccccatcccctcccatcttccccaccacgcggccgttgggtgcagcgcgcgcagcaccacgcgggcaggcggcgaggagcagggagcgtgcgcgcctcttgcccgcccgcgggccgcaagattccggaggtggtccaccccgtggtccacctacctctgcttctttcccttcgcccaaacggcagcccttccgcattgcttcctgtcctttagcgcgcgcacccgtcacctcacgctgcacttctttcgaccccctccaggcgaccctgtatttcccttttttccccctttactcatccttccccttcctgccaccattctgtctctcccatttaccccatcgtggcagcacccactcccctttccctccctgtcgcatcttttgttccctgtggcgcgcaggtcaccctccgttttcttcatggggacgcggtgctggcgcgcagtttcccgcagaaatcctgtggaggggtctgagtggcgctacagcttcagcttgcattcctctgtcagccccgtctcccccaggtcctctgctcgcccttaccagttgagccgaaccctttggattggtaccccatggaacaccagggcccccgagaagcgtggcgctggcggtgccgacctgctgctggcactgccgggccggggcgccgcagtgggcgggtggcctgtgggaggagcgcaatgaggcctgggggtggccgtgtgttgccatcctggcctctgccacttgagggtgaggaggaaggaggcagtgggctttgctggaaaaccgtaacagaaactgcctggccctcctgcggctaactgcatgcaagatgggtgtggccctgcagaaggcagatgacttcttgaagcgttccgagggagaatgaatctcagtagtaagattcatttctcactcaaaacagtgcttcattataaattactgtccttttctatgccagtgacagattgcatgctgattgagacactggataaattgcaaacaaaaatggacctttgtcacaggccactaggaagtgagatggagttagcatagcatggatcctctctggaagggagcttccagccctaagggatggctgggatttatgagggtgcttcagaagccggtatccacaaatgttaacccggttgctcgctggtgaaataactaccgtgaccctctattgatacctggtcgccgtggcacttcggaatcactccttcagtaccctttatccaagaccatgtggtgttaaggctggtttatggtgaatcatgagtgtatttttgatactatcagtaaagaaggcagtatttttctttttccttgtaaacctacatcaagatgactctggaaaaagcgatttactgcacctgtccaataaataacagtgtgtgtgtgtgtgtgtgtgtgtgtgtgtgtacaagtttaaggcccaaagaacacttttctctgcctgtagaaagcaaattgtgtgtaggtttggagggcttgtttccaggattctggagtcttgaaggttgtcaagactcagttcattagggagcagtcacaatgtgaaaagagcagagcccatttatttgtccaaagtgagagtacataatgactcaattaacagcataaggcaccagccctgagctggcaaaagagaaggctccaaggggaactttaaagcctggctgggtgttcttcatcaagacaatcatgctgcgtaggttgtagtctcctcatttatgaaacagtgatttggcctggatctaagattccttatac

SEQ ID NO: 3 - DNA, 7001 bp, Homo sapiens
  misc_feature: Human FABP-7 promoter and coding sequence
aatcagagcaaataagctactatttgaaagcaaccattcttaggtatacagccattatattctgaataatgtaaaataagtcaaaaatgtcagtatatggagttatagaacagtcgagtggcttttattcaacatggtattggaagtcctagccagggcaattagacaagagaaagaaataaagggcatccacactgcaaaggaagaagtcaaattgttattgtttgctgatgacatgatctcatatttagaaaaacctaaagactcttccaaaaaaaaaaaaaaggttagaattgataaattcagcaaagttgtaggatactaaatcaacgtacaaaaatcagtagcatttctatatgccaacaatgaacaatctaaaaaagatatcaagaaggcaatcccatctataatagctacgataaacaaatacctaggaatacacttaaccaaaaaagtgaaggatctctacaatgaaaactataaaatactaatgaaataaaatgaaaaaaaacacacaaaaaggaaagatattgcatgttcatgaattagaaaaataatattgttaaattgttcatattacccaaaatgatctacagatttaatgcactctctattaaaataccaatgacattcttcacagaaaaataaatcctaaaattcatatagaattacaaaagactccaaatagccaaagttatcctaagtaaaaagaacaaagcaaaaagaacacatggaagcatcacattacctgacttcaaaagatactaaaaagctatagtaacaaaaagtgcatagtactggcataaaaacagacacatagaccaatagaactgaatagagtacacagaaaaagtttttatatttacagacaacttattttcaacaaaggtgccaagaatatacactgtggaaaggacggtcacttcaacaaatggtgttggaaaaactggataaccacatgcagaataatgaaactagacccctatctctcactgtgtgtgaaaatccaattaaaatgaatcatagacttatatataagacctgaaactatgaaactactaccagaaaacattggtgaaatacttcaggacattggtttgatacagattttttagtaagacctcaaatgcacaggcaaccaaagcaaaaatggacaaataggactatatcaagttgaaaacctcttcacaacaacaacaacaacaaaaaaaacaaacaaaaaaatactaaacaaagcggagagaaaacctacagaatggaagaaaatatttggaagctatccatccagcaagggaagacgtaagagattcaaatgattgaatagcaaaaaacaaacaaacaaacaaacaaacaaaaaacaaattatctgattgaaaattagcaaagatctgaatagacatttctcaaagaagagataagaatggacaacaggcatatgaaaaatactcaacaccactaattattagggaaatacaaaccaaaaccacaatgagatatcacctcatcccagttaaaatggctattatcaaaaagacagtaagaatgttgataaaggggcaatgcttatacactgttgttgggaatgtagatcagccactatggaaaacagtatggagattcctcaaaaaacttaaaatagaactaccatatgatgcaataatcccattgcagtgtatatatccaaaagaaagaaaatcagtatatcaaagagttatctgtgcttccatgtttattacagcactagttacaatagacaagatttggaatcaacctgtgttcatcaatggatgaatggataaagaaaatgtggtatgtatacataatgaaatattattcagccataaaaaaggaaggaaatcttatcatttgccacaacatggatggagctggaggatattaaacgaaataagccatgcagagaaagagaaatatcactttttaactcatatgtgggagccaaaaaaaatcaatctcatgaaagtagtgaatagaatggtggttacaagaggctgggaagtgtagtcaggaacatgggatgaagagggaatggttaatgggtagaaaaacacagttaggtggaatgaataaattctagcctttgataccacaacaggtttactatagttaacaataattcatagcatttttcaaaatagctagaaaagaatatttagaatgttcccaacaaaaagaaatgaccaatgaggtgataaatatcataattacccagattcaatcattactcatgtaatgtttgcatccaaatatcgtatgtacctgataaatatgcacaactattatgtatccatacatgattaattaaaaaatagtcacttgactttttctaaaatttctggtacagatatctgtatggcaacatgtattcttttctgcctattacaagattaatatgcaaccaaaaaaacctgcctctaaattgtggggctttcatgtttttgataaggtttaaaggaatcagaattaatggaatatatgctctacgaataaatatgtcaccatctcatattctttatctagagagtggaagaaataaatgggtatgcacaaatatttcagcacttgtgatctataaagggctaagatgaagatatctaatacatggttcctgctctcaacaattttatgttcttagaatacaaaaggaaaatgatttattatattttagaaaaatacttacacatcctaccactttaaacgtgttttattagatgaaacatatagcaataatttgtaacaaaacaactgtttgagaacatttactagagaaatagtttgcctagatgaaacagtcatcggaattttagctgtaaactggaaataaaatcctaagcacccccacatactgagcagacccccttttggccaagggatgcccgcctcaaaaaaacccttaaaacctgaattcctggccataaaaggaagggaagtcggacatacttctgactttttggagtttagacacaactgacaggcattcatgttaaaatagagattataaaactgacaaaacagactctttgtgtcaataagacaccaaactataaacaagatctaaggtaatgcaaggcaagaattaggccattcatcttgccacagagcatccttttgttaatataaaacatttatgtccagcctgggcaacatggcaagaccttgactctactaaaagtacaaaaaaatagccaggcataatggtacatgtctgttgtcccaactactcagagggctgatgtgggaggatcacttgagtcgtggaggttgcagtgggctgagatcgcgccactgcactccagcctgggtgagagagcaagaccctttctccaaaaataaataaataaataaataaataaatataatttttttatgtgcactccaagttttagacagagccttattcctttaaccaagtgcaaattaaagaatctctgaattcatctataagctgtaagccctcagcttcaagatatcccagctttttgagctgaaccaatgtatgccttccatgtattgatttacgtttttgactgtacttcttggctccctaaaatgtataaaaccaaactgtaatctgaccaccttgggaccactttcccaaggcttcttgggtttgtgttttccctgggcctcagttgctcatattggttcagagtaaacttctgtaaaacattttacagagtttggtttttctgttagcaaagcaaaagagataggccttgacattaagtttgattttagtggtaaagtattagaaatgattcagaggtttctggcttgtacagttgggtgcatggtggtatcagtcacaaataaagtgaaaattggaagaaaagcagttgtgaaggaagactacagtttttgttaaacaagcgcacttgagacaccgaagaagaatcatgtagccgccacctagcgtaggaggccttctgagccttctctgttggatctggtgtggattgggtagaaatgtgctgggctgcagtttaattgggaggggagagagaagggcagggaatatcaagtcggattctcctgctctgtccccaggagaggcaggtaagctgctttcattgctggtcagcactagtaagtcaggaaggaatctctttgggtgaaggggaatttcagagacttcctttaaatactctacttagaaccctcaaggtatttgccattcttggggaaccctgagccagggcaaactgaaaccaaagcccttctgattcctaccgctcctccagttcctgcccttccccatcactaactcccaacaaatgtacacagattgtatttgtgtagatgtggcctcaggaaatcaaaaggtcctagaatgcaggtaatttaaagtcttactaaataatcttctgtccagtttttccacatgggttctataatctattcaaggagatcggggggaaaagcattcttttatttatttttgtacctcattagtaagtcctaaccatactcttccttaggtcccttttccaactcagtctcgttctcacatttttagaaaggacaacgggatctttaaattctctcggtttgtgttgggaaaggacccaaattagtgtgcttcctcattgacagtcagagtagccttcaagcagtatatccagggaaggcatttacacctcctccccgccctctttttcagtttggcctactctagcctgcatttcgctctccctgtaactatgagagaaaataacacacactattcactctcctttgtctgcaaagtctggcatttcagtggttttctttaaggggggtcctcatttgtgaccagctttgtcattaacggaaatcaatctgaatgccccattgtgcatcttttttctttccttgcagtctgagattgcctttgccaagttttttctcaccgaacctgaaagcccttctctcattttcccgttccctgcctccagccccattgaatccctgccgagctttctcaggcataagggctgtagtgtgaggattgggaggaactcgacctactccgctaacccagtggcctgagccaatcacaaagaggattggagcctcactcgagcgctccttcccttctcctctctctgtgacagcctcttggaaagagggacactggaggggtgtgtttgcaatttaaatcactggatttttgcccaccctctttccaaataagaaggcaggagctgcttgctgaggtgtaaagggtcttctgagctgcagtggcaattagaccagaagatccccgctcctgtctctaaagaggggaaagggcaaggatggtggaggctttctgtgctacctggaagctgaccaacagtcagaactttgatgagtacatgaaggctctaggtaggtaacaataagaccggctgttctcttctcaacgtgcagctttagatatttgcagattccatttttcactcatcaggtcagcaagaggcaaagacagatcacattgccctgatcagatgctaggctatgcattttccactgaatggatttttaatgtggcacttgcttgccttcttacccagggccaataaaaacagaacatttgctattccgtggagaatatgaagcttctctgacttttttggttttcttctgcatccacttattatcacagtagtgttttacatgattaatgggctaatcatgttctaatgaaacttacactttagaacgtggattccagaatcagaaagcagattcttgctttgtgagttattttggaagtcgctgcagactgtactgttccctttgctattttaggcgtgggctttgccactaggcaggtgggaaatgtgaccaaaccaacggtaattatcagtcaagaaggagacaaagtggtcatcaggactctcagcacattcaagaacacggagattagtttccagctgggagaagagtttgatgaaaccactgcagatgatagaaactgtaaggtgagaaactgcttcttcttcagagtggggatggggagaggggaataaagataaagatttccaatgcatgttttttttaagatgttggaaagtgtagaatattttcagtatttcaatccaaggagaaatacaccaaagttagtttaatgtgcatatgttatattttgaacaatgggtacttgctatagcataaatctcagttgtatttgtctcttaccatatactgttcacattttatatcaaacttgtatttcagaaaatatttaataaggtttgtttgaaagagagattgcattttgtttaattccccaggttgcctcagattgactaaatagacagtttgcctcattctaaggctttctttagagggacgactagctgcaaagcactggtttaggatttttgtttttaatatattaaatttttgtatttagatgtatactttttaatcaaattatgaattcaaatttacataatagaaaatctagaagtaatagaaataacaataggataattaactgatcatgatttattcaattatacaactctttcaaaaatcacatcgtcttccgtatcagaaatctgaattgtatttattgctatgttctgcattttgttgttggtctcagtctgttgttagcctggatggagacaaacttgttcacatacagaaatgggatggcaaagaaacaaattttgtaagagaaattaaggatggcaaaatggttatggtaagtaatgacaattctccattcttccttgtttttttctcctctccgcacacctctcacctccttccttcttcttccctcctccttccattcttcctccttccttccttctttaataatacatcactggcaaggttctttaactatatgtaaaacaggggaaacaaaaagatcccagttaattttcttcttcagctcaacttctttgattgtagaaaaagagaaaaattcatgtgtagttaaaaaaataaaagttgccattgatttgcctaaaactgtgtcaaaagattttttgtaggataactat

SEQ ID NO: 4 - DNA, 7001 bp, Mus musculus
  misc_feature: Murine FABP-3 promoter and coding sequence
attgagtacacctgtacgcctttattgtcacttctctagaactcttgaggaacagggaaacagatttcctcgtggcagaaggaacattccctccgccgtgccccactgccgatgttcccctacgctgtgtgggcgtgcgctcacctctggttgctgtctccatttttcgcttggatccctgctgagctcagctcaagtaacgggttttttgttttggtatggtttggttttttgagacagggtttctctgtatagccctggctgtcctggaactcatgtagaccaggctggcctcgaactcagaaatctgcctgcctctgcctcccgagtgctgggattaaaggcgtgtaccaccacgcccggctcaagtaacagttttaaaaggactatttagaggagagaaatgcagagatgagggaacttagtcacagtatctaaagccggctatatggcctcagtgagaaacgctatagatcctttccttctgactcagagtcgtaaagtaactttacgactggacaagagaaataaaggaaatttctatccactgaatattaatatgttgggatttctatttatttatgttcatttcccccacctcctccaatgtaagaatcaaacccagggcctcatttaggccagacaaatgctctgtcagtgagctatactcctaggctcaagctctgagaatttgaaggccttcctttatgaggggaaaggagactgggggtcaagaggcttgcctcagtgttctacacagtaagaggcaggtctgggatgcaccaagcagtcctctttccaaaagcagacatgccactcactaagaggggtggcagagcagtggagaaagctcagactcgctgtggcaggcactaacagactgccgtataccaccaacgccagaagagctccctgttgtatggagtagggaagtgcagaaggagcatctgcatctctgggttggtggagccctggctcagtctctttggcctctacctctaggttcttccccaagctagtggacactcacacatagaggcatcaagatggcaggaaggcccctccccctgaagcagctggagatgagttgagtcgggtgcttcctcttccccctcccccaggggccacagatcagccagagctacagcccatcccagggctccctcctggttactcacctctcctcttcctaggcccagcctgtggttgtcatggaaacatagaatcactatagggggaagactgggggagatgctgctcttgaagggcacggatgcctgccagatgtgatagccacatctggatctatgagaggctagaagggtgaggaaagagacgagacccacccccagcttctggtgtaaggagcataaccggggcaagttgaatgtattccctcagagaaggaagtgtgagccgtacacacttggaagggagaatgagctgaagcaagtggtgcaagcctgggaaaactgaggcaaagcctacctgggctatagcgtaagttcaaggccagcctgggcaactaagtgagatcctgctcaaattaaaacaaacaaacaaacaaacaaacaaaaataactgagggatggggatatagatcttcatgggaaagcatttgcctagcatgcaggaggtccagtggataaatgtgtttgctgtgagcgcctaatgaactgagttctagtcccaaaacccagagtaaaaggaaagaagaagcctgaaagttgtcctctgccctccacacactcatgcctgcagctcacacccaacggtcatgcgcacaaacaacaacaacaacaataaacctagcagaaggaaagagggtgtggctgaggatcaatcctcgtggcccaggccagcgctttgcacaggtctatagctcaaggtctcaattcaagatcctgtgctgctcagctcaggcagggctcagcacaagaacacaggatagtcagcaccgtgtcttacggccctgaagcctaagacctggctgcccttcaaggctgtaggggtaaacatcagccagatgtgacaggtgtcttctgtcactctccaccattctcttgagacagggtctcattagcagccctggcttcctacttcctacttcctcggggatagaattaaagaggttaaaggttttgttctttttcttgcctactttatttttgaaacagggcttctcactgaacttaggagcctgccatttctgctacatggacttaccagcaagcacccttacccaggatccacctgtctgtgtgccctgttcagtgctgaggttacagcatgaacaaccatacctggtttttatgtgggtgctgaatgtgtgcatggtaagcaccttaccccactgagctatcttatctcctttcagtggctattcttgaaagcaaactggctaggtgatggtgcacgcctttaatcccagcatttgggaggcagaggccagcctggtctacagagtgagttccaggatagccagggttgcatagagagacccagtcttagggagaaggaaaaggaggaggaggaggaggaggagggggagggggagggggagggggaggaggaagaagaagaagaagaagggagaggaggaagaaaagaaagaagatcagggccaggtgatggtgcatgactttaatcccagcactcaggaggcagaggccagtagatctctgagtttgaggctatcctggtccacatagtaagttccagggtagccagggctaaataaagagattctgtcaaaaaaaaaaaaaaacaaaaacaaaaaaaacaaaaccagagtttttaatgtattttctctaatgatttccaaatatctcccatttcaaagatgaaactgattccaagtgacttaagtgaactgctcaagaccacatggcatggctagtatagctgtgatcgggtctagaacacagatctttttgccagcagctgtccgccagccccagatctccagcactacattccacactccatgtaagcagactgctctggtccctgggtctcaggcctctcaggctttgagagaagaggctccttgcctaaagcctgtgaccttgaatcagaaaccatcccatgccctccctcccttggcacctgctctatcgctgaggtcaccagggctccaggcccagccctgaggcccgtccctgtagctttggcagaattggacctctcagccttttggcaactagactctccaaaggaatcaagacaggggtgagaggacttttcctcactggcctccttcccagaacagcccatgggtgggagaaaggaacttccacggggacagaggcccaggaaggaggcctcaccacactgcaggtatactggtcaggctccagagctctggtaaccgttccggaatgactaccctgcacaaccttctagaggccaaagttctgtgcttaccactccgtcccgtgtagagagctcagaaattcatttatgtccctcttcctctgaatccccaagtcaggacaggtccctcctagagtcactcccagagggagtactttagcctgtaccttgatccatgtggacttctccaaagcagggcccgcacaaaccaggagcttactttgagagagtatcttgccttggctcagactggcccccaactcaggaatctcttgcctccaggtgcaggtgccgtattaactgttggctcaagccagagcagcggcacacagctgtaacccaggagacagaggcaggagaatctttagttcaaggtcaacccctgctacacattgagatgctgacttggtaataattaaacagaaactgctgaactaaggaataggctccactgaggttcccttactcacctgtaaaaaggggatgataccacctaccaacgaaaaagttgagtgtgaccatgccctgaagtaggctacaaccatcaatagtcgggtcttatttaataacgtactttaaggtgacaagcagtctagtggcagaagtcaggggaaaaaactgacttcagcagagggtcgcggctttccgggagttaaggtggccgaggccggaagaaccctctgaatagacaaattgtcttcgcggagtgaagaacgaccctggcacaagctcagaggtcagtaaataaagcctgaagcgctttcaggcagcggcgacgggtgggactgcggagaaaggcgcaggcgggagacattccgcagggaggggctagcacgtgtggggctagcatgagggaagcaaggtcacgttctccgccagcaggtgaggcgctgggcagctcagccatccgcggtgtccaaggcaactcttttccacttgtctggtaggagcaagagggctcaaaggccactagaccatgctctctgtccaggctccaattcttttttacttacggcgaccgcgtcattcctctccgagcctctgagcctcttctacaagaagaggacataggaccgttgagatgggtttttgggtaaaggcccttgctgtcaagccttgacaaccccagtttgatacgtgggacccacacggtggaagcagagaagggactcccgcgagttacaacgaacgccccagtctcccaccccttccccataagtacgcctacacgagcatacacaatataagaataaaaccacagcgaattaaaaaacaaggcggcagaaggatcaagcggcgtttctccagcgtggcaccagctcaagggcgagtttcctttcagtatggccgggggatgctctacttgggttgcgggaagcgccccgcagccaggccagggatgggttagatggcaccaacaggaccgcgggcgccgctgacgtaggcgacgggagggctgtgggggatgggccccagccctttgcgggagtgcaagccccggcttcctatttcgggagcgaggggtgtgggccactttcatcatgtgatgcgagggctatttaaagaggctgtccagccgggagctgcggttctcagtgcctgctcgcctcctcactcatcgcaccatggcggacgcctttgtcggtacctggaagctagtggacagcaagaattttgatgactacatgaagtcactcggtgagcgaacgaacggcgcaggatctagggtcaggagggccggcaaggcggtcttggcgctgagctcccagggggagtgcccccatgtgcctcccgcaagctcctagccagtccagacagggaatactgaggtgcggagggtggcctgggctgaagccactccactccaccccaccccaccccggcctcctgggaggggggtgtcgcggtccaagcttggcgagcctcgtagctggaggggaagggtagaggcagctgtggccgcagaggtccgggatgggaggctttctaggaagcagtgtaggtgatccggaggtggaaaggggagggaaagaagggcgggaggctggccgcaggagaaggcaaagaggagcatggtggtccagaaattgaattccgaaagggaatagagcagataggagtgtacagagcctggaggaagactaaagaaaatcagtgaattccatctgggaagaggtgaagatacagccaggcagtcagcaacaagccctacccctccatgttggggtagtgaagaggcctctctctggaagatgccctggttctcaccagcctgaccttcacctacagtgtgtgcagccacccctgggatcagtcggagacgctgctgctagagcagggcaagacgaccactacacataggcttcccggccgcaccagtcggccaccggatcagtgctggggatagggtgaagaaagcctgggatcgagcagagggtgtcagaagaaaggtgaagagctatgaagggagagtgtggcttggggctggggaaattgtgtggtgtgggcggtgacacaacgcctttaaccagcactctgggaagcagaggcaggtgaatctcccgagttctaggcctggtctatacagagagaattccaggacagccaggactacacagagaaaccatgtcttgaaaaaaaaaagaaaggaagagtcccatgatttacttaataggaagacagcttgggacacatgagctcatcgcctcataggaaagcccaggatttctttttgaagactgaactagagccttgtgcatgccccctactgctgagttatactccccactcacacacacacacacacacaccctctttttactctgtgtaacaggttctcactaacttccccaggttggctttgatcttgtaacctgccatctcggccttccaaatagttgagaacccagaactacctagagttcttcccatttcaacagtggggaatgtcacatgaaccacttatccaagacggccccagcccttcctttcttgccttgagcttagataaagacctctacctgcggagtccctggctatatcatcctggtctaggaggctggggcagggaaaacaggactgtgtcatgcctgagctagcttccactccgtcttccccgggaaggagggctggaatcggacatgttgagggatgtgtgtagttgcctctcacctacttccagctcttctctgaaacaggcccacaaagcaatttgtccttttggtttggggaatggaacccaaggccctttgcccgtgctaagcaagcactgcgactgctgaaccacatctccagacagggctcccaccggcacctaccctaccctgaggctctccaggaaggcagctggtcttgtcttttaagacagggttttactgtgtatccctggctctcctggaactccctaaggagaccaggtcctgttgtctctgcctctcccgcactggggttagaggtatgagcccacacccagctagttggctgtcaagttagggagtactagattacctgggcttagttctgtttcactgagctgccgctgccttcctagacttctttttgcctcagggcatgttgtcttcaggccattgttctgtgggtgctgaacccagctttagtcaggggac

SEQ ID NO: 5 - DNA, 7001 bp, Mus musculus
  misc_feature: Murine FABP-5 promoter and coding sequence
tcctacagtttccctttggaagcagctctgtggtttttcttataggaaagccctaactagaagcgaggtaaacaaagcaagtcaacagtagaacaagccagacttaggcgagttcacagatttgctatacaacttcagggaggcattaaactttaacagacgcaggctcaaacctgctcacctccacatgccatttgcagcctttgctcaggcctcctggaaagtggacagataaacagctgcccctgccagatcggaaaaagttcaatgactgaatcagatacatttagtcaatttgatttgtttcctgtttgtcttctggagtcttgaaaggatgagttagacagacagacagacagacagacagacagacacaccaaatcagctgatactcagttctcatacatgagatagtatttgaatatgacccatgcataagcccccatagactctgtctctaagtaccaatgcaatgtaaactcaatgtgaagagttatgtataatattataatgaaatttgcaggaaaatgatgcacctgaaaagtgtaatacttatcaaggtgacctaaactcagaaaataaacctcaaattttcttcctcatatgcagatcctagcctttaatgtatacatgactgtatagaaacagtggtaagggtaagcatagtataaaacctaggaggacaaaagaggctcatgttaggcgatgaggagggtatggagcaggcacgagcacacacaagtcaccaggaagacgtaaagctcgtttagttttatagttttgactccaccatgtttctcttctttatttgtggtgggtatttaagttgaacgttctgggtattaaagtagtaagagccattgttatactccaaaggaacccaacatgtgttattggtgttaggctttccgattctattgatcatgtgcacagtaggagtcagtcgtaactaaacagctggtgaatacatgtgtcccattgacccacttagaaaaggcaattttaaaaaaaaatttggttttcccacttcctattccaaactctttaaatcattctgttatagtgttattaaaatgcaaactatgttgttataataaactaaagtatgatgtttaaatataaaaacaagaattacactgtgttgtttaggaaatagcaaaaagataaaactctccataaattgattagactcatttttcctcaaatactttttatctacagtcagtcaagtgtctatgtgtggaatgcagagatggagagagcagatactgttctcacataaggagataatcagaaccactgtaagagtgtaatataaaaatatttcatgtgctgaggtgggagtggcctcttgtggcaaggtcacaggtgtgggtttgggcatcttttgcaaccttggtatctggagcacggtgcctggactagcagtcttaacgccagtaaggacttagtagatgagaagagtccaggccctgctctggacctactggatctgaatatgcagttccaattgtagacttgtccagtctctcactaaaacatgagaagtgttacttcagacacatcccactttatacttagctcttctgcctttcaaatgtatcttgccaggtatttagagaatacttaactgtctaaacctgaattacttcgggtattctgtgccataatttctttaccgtggtgttctctctctctctctctctctctctctctctctctctctctctctctctcatgagctacattgctaagccacctatacttactgccctcaggaattattgtaaaggatgaggagccgtgtgagagacgcacctgcttacacaacagctgctagggtgtctgtaaaggaaatcacattccgtgttccaaagctacctacctgtgtccacaaatgagaacacaggccatccgtgctaatcaaataaagcccgggagaaatacttcgctactaccactaggtggcaatagagtgatttttcagcctttcccttcagggtcatgaactgagtcgcgcatagatagctcagaaactgcgcgggcctcatctgttctcaggtggccagaggaaaagcctcaaggaacgctcgagccttcatttctttccacccgtggaaagccctgcttaagcgggccattgacacgtccctgcagctctagtggaggaagagctggaggaagtaacacaggtgctacggttttcaaaggagagacctgcatgcttagagttgatttaggtgaatcaagagtctaaggcctttcctatggattcttgttggcctcttcaaatctgacccttaactcgggctcattccgaaagaaatgtgcacattgttgaagccatctgaatgtgcctaagaaaatactaagcttctaaccctagacatggccagtgggctaagtgtgagctgtggtcagggacgcatgaaaatctgaatgtaactactaggtttgaaggcggcatctatttcaaaggcaggctttcgattgtgtggagagtattgagcatgttcgaaagaacaaaacagcaacttcgtagtgagatgggagccatactgtcagattaatgtatatgtcagaaacagtcacagccaatatttgcaatgtggactgatgtatgaaatcgggaaacgcaggacacagtgtgtgaatgctaaagactgcagtcgtggttctgtgtacacatattcacataacacatgcacatggacacatgcacacatgcacacagacacacagaaacttgtaataaaggggatgtaggccgtggaacttcttgcagcatggtagaggcttgtcagattttaattgtgtgagggatgatacaataattagtgtcttggcagggtggctgtggcctcttctcttagcttccttataagcatcatgaacaaaggacaggcaggtgtggtcctgtttcacaagatctttcgtgagaaactcctcagtgcgattccagttctactactccgcagcaccggctctcttagcctcgtctcctctcctgtgaaactgggataacttcctcaggaatcgcaggcattaaaattcatccgtgcaaatgccaggaaggaccacagagggagcagtagaaactcaacaagctgaccttgtctccgtggtgatagcccgtctgggtggcgacaacttcatttcagcccatgttacctccaatgaggtgtgggagcaggcaggaagattctgtaaaaaagacagtctgagaaaggtcattccacacacagaagggaactgatggcctcagtcagtgggatgtcgcagctcaggacacatcaatatatgtatggccttagtagagaatgtggcacttcatgtatgtatggcatattttgagctggtctgagaagctgcaggcctttggatagctttgaaaggacgtactttagacaaagaaaatcataagtataaattgtcgtgcatttgtgtgtgtttgtgtgtgtatgtgtgtgtgtttacgggcctgcatgtgtgcacatgtatgtggaagctagtagttgacatcagctgtctgccacgacaggatctctgactgacccggaagcttgccaatgccgggtgggctgtaccagggagctgcagttctaggtgcagacacagcagcagcttttgtgtgggtgctgtggatctgaaggagtcttccatcctgggttcacttcctcagctgcagggcagcgtgactgcagtaaattcacttagagttattgtatatatccaacctagtaaaaaattatggctcagctgccctcaaacagacttaggacatttacattggcctgtggttgggcaaattatctaatacaaacctgaatcctaacaaagcatggactatcacaggtggcttactaaaaatgacactgaaagtgaggatcagtaactgtgtaatgacaacttccgcaagggtgttggccacacctgtaatgtcagcatgtggtagctggagagtgatggtcaggagctactagctgcatatggagttagaggctagcctgggctacatgaccgctccataacctttactcaattcctcttctcactgtctcataaaatgtgctccctttgtctccctgtatctctccctgcctcccctccccacacactcagattctgtgactggcgatggcatctaactgtcagcggattgtccttggagccttatgttttgtgggacttcagtgcatatcaaagtgataattttttttttatagtcagtatattctaggcaatttactaattcatagctgggccagccaccctcaaaggatggagggaagtaaaagttttgatccctgatcggggaatagaagctagtaagatttttcaggaagtgacagttacctaagctgaaacttgctagtgtagtggagaaaacaaacttgtagagcattgtaatctcccttagagactgctgccaggattgtctagcatgcacagacagacaaccaagaggccccagtgaggccccaggaggcgggagggtggggtcagaaggttctttcatgagggagtaggatgtagggctaaggagattgcagtttattaattcagtaatcagccattcaattcctatagcgccaagcgatggctaatttgttcctcccaggtacttatcaaaagcaaatggaggcggcaggacagtgggaccacggcgcgcaggctgtcctcagatggcgcgggtgggggtgggggtgatccctgggcccacgggactgtgccgcagctgtcactaagggaggcacagggcgcgccagacgagtcgcagcttgcaggagctgcgggaaacgtgacgtcccagtccggcgcagccaggccagggtggcaccttcagaccccccagcacagcccaaggcgcacaagtccgggcgtggcgaggggcggtaccaggctgaggcgcgcctcctattggctgcccgacaccacccccacaccaaggacgatataagcgcagatggcgccaggagcggaacagcgccctcgccaccgcgtctctgctgcttttgtgctctccctcccaccatggccagtcttaaggatctcgaagggaagtggcgcctgatggaaagccacggctttgaggagtacatgaaagagctaggtgagattttatcgccgaacccctccctggctcctcatccttgtggggatgtgttggttcccttccaccatgcgatcgggggagctgcagggagcaccttgcggcagttaggatggagtgcccgcctttgtcgccggctccgcacgtggtgggtcaccccgcttctccccttttctgcttcttgcccccgaagcagcccctcattgcttccggcccttcacaggtgcgtgccagcggtactgctgtgagccccctacaaacagcccctcagttgcctcttaatgttatccccacttctcttcctatcctcacctgtgccaccgtcctgccccatcatcctcccctggcgtggcagcactcactctccttttccctttgccccatcttttgttcctcgatggcgccagtcccctccaccccatctccgcctcagagaggacaccgcgtggtagggcgtggtttctgcagaagctctttagaggctatgaatgatgctgcttatatccctttctctgcagcacgtctcaggtcctctcctcgccgcactggctaggcgtggcgcccgcggcgctgcacggtcgacagctggtggcatgtgggcgaagtgcggagacatccattgtgcatggttgttgtcttgcgaccgttctgttctacttctttctcttgattctggagggtgaggagcaaggagacttgggcctggctggaaaactataatcagaaactgctgttgctgggccgtggctgccaactgctacaagatgggtgtggctttgcagcaagcacttgctccttaaagcctaaccagagagtgggcaaggttcaaattcaccctcactggaaatagtgcctcgttgtctgttgccagttttgcctactcttaagatagggatgcattggtaaacacagaacagacaacgaaatcgacacttgtcacagatgctaggaagtgggatggggttaacggaaaggtactctctcggaagaggtactgctggtaggaggaggcagcttcaggcgctgctgttcacagtattcctccctggttaagaaccagccttaattcttttaaacattcagcacactatggggctaagtctggctcctgatgaatcctgtttgtgctaacgctattgtcagaatgtggatgcattaggggtttgtgtctttccttgtccacagtggaaagccgcacaggtgtgagtgagagccagtagagagcactgtttgaaagcagctggtgtttatgctgtgagtggctagttctccagattgtggccttaggaaggttataggccttacccataggttctgggcaatgtagaggggagcacagcagaagactcacagggacacgggagcctggctgagcatcctgcttggacgagtgtctcctttcttgtgaaagtatcccaggttccttatgagtgttaccatcaaggacacggacataaaccactgcataacccagaaatgtggaactccatactgtgaataaacaaaccacacagcataaaagtcaaccagatactcaaatcgtacagtgtttccagatagcagcacgtcaggttcctaacatacagaacaagccttaccatggaagctcacatggtcacacagctttgaaatgtttgttctttccctgggtgtgttttccctctacttattgacaccagaggcatgccactcacttaaaatactgtgctgtttgcattgaaagcttattttaaagcatctaccaactgttttttttttgtttgtttgtttgttttttttttttttttttttttacttacattgttgttttgagaacacggtctctctgcagcttaggctgactttgaactgttgaccctcttacctccatgtctggaatgctgctgga

SEQ ID NO: 6 - DNA, 7001 bp, Mus musculus
  misc_feature: Murine FABP-7 promoter and coding sequence
acttctcctcactaaggaacagatgggccattgtttcccccccttcactgcctcagtaccttctcaatcaaattcagttatcagaatcacattaaccctagtcttgcaggttctgatcttccttactctaagcaactcaggcaccaagcagttttagcatcttggttgtggagatggtgcaagaaagtgtaggtaagatagacaggtgacctaatttcagaaaagggggcagggaagagggggtctagttctttctctcttgccgattacaggagggctgtgtcaacagtacaatcagcacaagtcatatttcatttcaactatgaagatgttcccttaggattttgtataatttggtgatgaaaagaaattccagcagagatcctgaatgttgattgtctctctcagagtctactcctgggaattctgcaggacatatcagtcaggaacttggttgaccttcttctttttgttttttgtttgtttgtttgttttgttttgttttgttttgtttttttaaagtggtgaaaatgtatgaccattctctattttcttaaaagggaatgcagttagaaaccaagctccctgagatccagccttgtgtggagattcgccaagatctctcaaagccctttctctccctttttctttgactacttaaaataagtagatctttctaatggattttattatttcccattggcttgctgacaatggagctttgcacacactctaccactgagccatatcaccagcattacctaccgatcatggaaatgattagaggtctcattatccctagttaacatgtacagaaattagctctagaaggtcagtcagtcagtttatcaccttttcttcttgattttacgatgcatgtaaattattacatcctcaacagagtcatacattactccaaggtttagggaatttggagaaaaatattggttgaatttgccttagcacagcaaatacagtttctgttctagaactgtctcaatagagattcaaatatgcccttgtttagagcagtaagtattctgtgtatattttattcagtttatattacatagtgagtaatttgaatttgctcagatgtgttggtttaggtagtcacaaaatagattgggtcttccaaaaattctactgtacttatctacataacagtattttcctctgtaaatgaatatgaaatttatggaatgcttcctatgaaaggaactataaaatgtatagagttatagttctatataaaagaattatatgcacatgtaattttagttagaataaacccttcctgttgtagtcttcgtctgcagaacagagaagataaatgtaaaaagacaaatagttcagccctctaagctgaggagtgtaacacttgccctgcttccttcaaatgtccagggcagggctggcttagagatgctttaagtttctgtctttaactggagaacaagaagggtttgcataagggcttaaataagaagtcaagatctgtgggactgtgataggcagcctattttagttgaatggggcttgctctggtgacccaatagagaaatctacaagggatgaaaaggtgagccatattcccagaatcaggagcctgtccataattctgttactatcagtcgtgcagacaatgtcccaagcttctggcattctggttagactccacccccacagccacctgactatagctcactataagaggggctgcttggcccctcctcgctctttagccctcaggtttcttactctctagcctcctcctcctctctctctctctctctcccttcccccatctctccacatggccatggctggtttctctctctttctaccttctttcctttctccctgcctttctacaataaagctctaaaaccataaactgtctctgatcatgaaggcctgccatgcttgaactgccatgcttaggctttctcctaaggagccgagtctaacctcccaccagaaggccatcctgtgctccagccacagacctgaccaaggactctcgcccgagtgggaaccatccagtgccctctcccccagccctctcctcctttcatccctggggccaagtgccaccccgggcccctattctgttctctgttcctttgctgtatccagcctgggatgtcggaggccgagaatctggtacctggggctgccccttgtctaccacccgctgtgtggggttagtggcttcctacagcagacacccaccgggggccgtgtggaaagcgtgaacagtcctcctccgaccacccgcccagagcactggatctctggcgggacacgggttctctcccactcccctctttcccccacatccacagccctgcaaagatcaggctttgcagacaccactgttgctttttgccgcttgctgaagcaatgatcaaccccaccgcgttcttccttgggccacgtctcctttgagctgtttgcagacaaagttccaaagacagcagaaaactttcatgctctgagcactgaagagaaaggatttggctataagggttcctcctttcacagaattgtcccaggattcatgtgccagggtggtgacttcacacgccgtaatggcactgatggcaggtccatctacggagagaaatctgaggatgacaatttcatcctgaagcatacaggtcctggcatcttgtccatggcaaatgctggaccaaacacaaatgggtcccagttttttatctgcactgccaagactgaatggctcgatggcaagcatttggtctttggaaaggtgaaagaaggcaatgaacattgtcgaagccatggagcgttttgggtccaagaatggcaagaccagcaagaagatcaccatttgtgactgtggacaagtctaatttcttttgactttcgggcattttacccatcaaaccattctttctgtagctcataagaacacccctaccccatctgctctcaatgtcctgtaatctctgctctcactgaagttctttgggttccatattttcctcattccccttcaagtctagctggattgcaaagttaagcttatgattatgaataaaaactaaataagaaaaaaaagtcaagatcagatttagaatttaaaacaactatacagaggtaccattaactaaattcaatttatatgttcttaagatgtatacaaaataaagtttgatatattcgtgaaatttttgttttaaccaagaaaagaaaaaaaaaataaaaaggatttttttgcaaaaacacatatccaccaagtagcttatctaaaatatacaaagaatatgtatatagcacagcagaaagggaaaaaaaaaccccaactttaaagtaggaaaatatttacatagtcactgcatgaacggtattcaaaacccagaaaatcacattaaaaaaatgttcagtgtccttagtagtgaggggaatgcaaatttgtgtttgaaaggttgctggtttccaagagatcctttgagaacagtgtggtagctgctgctaaagaatcaggcaaagccagaagttgctcacatttaggaggcttgtgtgctgggggttgggaggatgactgctaaatactggggaagcaaaatgagctaagtctttacagcaactgtcatttgtgagagtaaaccattagaaacggacggcaggtttgtggtagattccacaaggtggtggaactagtcgcatatgtagttcagacttgaaggcacacacgtatggggacagttctggtgtttacgataaacaggtatgctgccatccagctagcacagcactaggctaaagcgtactgagcccttgtcttccgtgggagctgcagagtgggatgcatgcgttgtgagctgaggctcaagctgcgctggcagaagagcaggggttgccttgtcagactccagggtctctttctctctgagcctgggaaagtgccactttattggatctataaagccgggggggggggggggggaggaatctcaaggtgaagaggaagttcacagacccctctaacgcctctattagaaccttccagctattctctcatacttgtacactgagctggcacacagtataggcaagttctattcgcatcacccctctagttcctgtctccctggttatgcaagcctcatatttaggtagatgtgaccttaggaaaccaaaatatcctttaagatcttactaactggttgcctgttcagcttttccacattgatcctgtagccccctcgaggaggtgaaggaaaaaaatctcctctttgtttctctaactcattaatgaattttaagggcactctgtaaggttcctttcccattctggtctggttcgtacattctgagaaacacactgtgtttgtgttgagagttggctccctagctacactgtctgtcacattgatgctctgagtagggacagggttcatctaggaaatatattttcactcacactctgtatcttttcctagtttggcatattctagtctgcatttggctctctgtttaaatataaaagaaaactaaaacacacccttcagacgcctatgtctgaaaaatctggcatttccgtgggtttttctttaaggaggccttcatttgtaaccaacaccatgctctccttaaggaaatcaatctcaatgccctattatccttcccttttctttcctcccagtttgaggctgcagttgcctttttttttcttatcccctgctgaacctgaaaaaccctctcttttctacagttttctgttcccaggccccgctgacttcctttagagcatgggggggggggggatcaggattgtgatgtgtgaactgggaggatcttgacctactccgctaacccagtggcctgagcaaatcacaaggaggattggagccatctgcccagcccctcccccacggcagcctgctggaaagagacaagttagtcattcaaatgattggctttttgcccgcttcttctctaaataagaaggcagcagcttctgctgaggtgcaaagggtttttgtgaattacggtggtgggtaagacccgagttcctccagttcctgcctattttcagctgactaggcggttaaggatggtagatgctttctgcgcaacctggaagctgacagacagtcagaattttgatgagtacatgaaagctctgggtaggtaaaaataagacgtactgttttcttcttaactctgtattttgatccttagaaacccccgattttacttaccaggtttctgaaagacaggctgagcacattgtgtcaattatactaagactgtgcatagcccactcaaataattcttactgtggtattttcctgccttcttgcgaagaatcaaataaagggaccatttgatatcctgtggataatgtgaagcttctgtaacttttggagaagggagtgagaaggtccttatttgtgcttaccttgtccatggctagtggagtaaccattaatcaggtttggacgaaatctacactttagaatccaattctggattcttagagtgacaagcagatttctgaactattttggaagttgctgttaactgcgctggctccctttgctgtttcaggcgtgggctttgccactaggcaagtgggaaacgtgaccaaaccaactgtgattatcagtcaggaaggtggcaaagtggtgatccggacacaatgcacattcaagaacacagagatcaatttccagctgggagaagagtttgaagaaaccagcatagatgacagaaactgtaaggtaagaggcagctttatccggtgaggatgggacagaggattcccaatctctccacagacatggagaaggggaacaagctttcagaatcccccttcaggaagaaggagccagagctaacattctagggtttactggttgtatcccagacactgagtaatttccagagccaaacttgcaggctatttatcctttttcacattttatagcaacttttacccagagagtattgaaccatgtttatttgaaagaaagaccacaccttgttccttctctctgatcatctcagcttgaccaattagtccgtcgcacagcaaagctttgcttaagcaagaagagtgactagttctgtatttttatttctgatctatgctggttttttgcaactggctgtatacatggcagctgaatcataaatggccacattttagaaaacagagattcttgaaagtaacagaggatgaagtgctgctggaataattatgagtgatgtactcagttatggccctctctgataatgctgtcgtttgacttctgtgaaaagtggacttctgcctgttaaccccctttgattggttgttgctctcagtctgtggttcggttggatggagacaagctcattcatgtgcagaagtgggatggcaaagaaacaaattgtaccagagaaattaaggatggcaagatggtcgtggtaagtcctaaggagtctgccttttctgtccctgcttcccttctaagcatcattcttttgcactcccctctggactcttcttccttagcccccacttttgctctcccccaaccccttattttcatctcagtttctcctgccgttcaaagtcccttaatatgtaaaacaggaggaaggaaaaactcagaactagttgttgcctcaggttggcacctgtagagttttaaaattatacatcaatttaccccaaaagtgtgtgtcaagaaccgtggtcatgggattgctgtgaatgtccagccttcaactggaacttaaatgtaagctactcattaggaaaatatttgaagataactcataggaaaacataaagtaacgaaatatcagagtagttacctattagagactgtaattaaggcattttaaacattttaaattttagtttataagagctcagtgcaaataattttaacaaaaggtgaagctttaatttttactagatctgtttgatgttactcccttacagttgttttcatatttaatgaatcagctaagttttc

SEQ ID NO: 7 - DNA, 1042 bp, Drosophila melanogaster
  misc_feature: Drosophila melanogaster CG6783 (CG6783), transcript variant A, mRNA.
attcgctcagaaaatttcaacagtgaacatcttgtgcagaacacagaccaaatattccaaaaatgtctttcgttggcaagaagtacaagctggacaagtccgagaacttcgatgagtacatgaaggagctggttctgccctcaaacaacatatgtatatatggaatgctgcaccgagataaggtggtgctttgccagatctatatttaaatgcatggaggggcttgccaattgataagagcggagatgaggatgagggaggagacgacggcccgcattgttcgttaaccttgagcacgacttttgacgttacagcggaacggaatctgcagggaacttcatggctcggctttattatttttaattaaatatgatatgatttgcattgtgagcgctttggggtccccaactttgttgggctaattcaattatgtgcagtctgccccatcattcatctttcggcgtcggtctggtgacgcgcaagatgggcaacagcctgagccccacagtggaggtgaccttggagggcgatacctacaccctgactaccacctccaccttcaagacctctgccatcagcttcaagctgggcgttgagttcgacgaggagaccctggacggtcgcaacgtcaagagcatcatcaccctggatggcaacaagctgacgcaggagcagaagggcgacaagcccaccaccatcgtccgcgagttcaccgacaacgagctgatcaccaccctcaccatcggcaacgttaagtgcgtgcgcgtctacaaggccgtctaagagactgatctaaaactataatatccatacgactacatgcattaaaactaactcagatagccaatattcttgttgactaacagtgaactaaaccaaatcaaatggaactgcacgctctgcccaatgttaatttataatcgcacccgtacacccgcagtcggtgtatgtgggaatactcctcgtactcggggaccaatttccgaatgcatttgaataaactattacttgaagtgcttttaaaacattccgactttctttt

SEQ ID NO: 8 - protein, 117 aa, Drosophila melanogaster
MCSLPHHSSFGVGLVTRKMGNSLSPTVEVTLEGDTYTLTTTSTFKTSAISFKLGVEFDEETLDGRNVKSIITLDGNKLTQEQKGDKPTTIVREFTDNELITTLTIGNVKCVRVYKAV

SEQ ID NO: 9 - DNA, 714 bp, Drosophila melanogaster
  misc_feature: Drosophila melanogaster CG6783 (CG6783), transcript variant B, mRNA.
attcgctcagaaaatttcaacagtgaacatcttgtgcagaacacagaccaaatattccaaaaatgtctttcgttggcaagaagtacaagctggacaagtccgagaacttcgatgagtacatgaaggagctgggcgtcggtctggtgacgcgcaagatgggcaacagcctgagccccacagtggaggtgaccttggagggcgatacctacaccctgactaccacctccaccttcaagacctctgccatcagcttcaagctgggcgttgagttcgacgaggagaccctggacggtcgcaacgtcaagagcatcatcaccctggatggcaacaagctgacgcaggagcagaagggcgacaagcccaccaccatcgtccgcgagttcaccgacaacgagctgatcaccaccctcaccatcggcaacgttaagtgcgtgcgcgtctacaaggccgtctaagagactgatctaaaactataatatccatacgactacatgcattaaaactaactcagatagccaatattcttgttgactaacagtgaactaaaccaaatcaaatggaactgcacgctctgcccaatgttaatttataatcgcacccgtacacccgcagtcggtgtatgtgggaatactcctcgtactcggggaccaatttccgaatgcatttgaataaactattacttgaagtgcttttaaaacattccgactttctttt

SEQ ID NO: 10 - protein, 130 aa, Drosophila melanogaster
MSFVGKKYKLDKSENFDEYMKELGVGLVTRKMGNSLSPTVEVTLEGDTYTLTTTSTFKTSAISFKLGVEFDEETLDGRNVKSIITLDGNKLTQEQKGDKPTTIVREFTDNELITTLTIGNVKCVRVYKAV

SEQ ID NO: 11 - DNA, 1661 bp, Drosophila melanogaster
  misc_feature: Drosophila melanogaster CG6783 (CG6783), transcript variant C, mRNA.
attcgctcagaaaatttcaacagtgaacatcttgtgcagaacacagaccaaatattccaaaaatgtctttcgttggcaagaagtacaagctggacaagtccgagaacttcgatgagtacatgaaggagctgggcgtcggtctggtgacgcgcaagatgggcaacagcctgagccccacagtggaggtgaccttggagggcgatacctacaccctgactaccacctccaccttcaagacctctgccatcagcttcaagctgggcgttgagttcgacgaggagaccctggacggtcgcaacgtcaagagcatcatcaccctggatggcaacaagctgacgcaggagcagaagggcgacaagcccaccaccatcgtccgcgagttcaccgacaacgagctgatcaccctaattccccacctaacacccagtcagcatggatcgctccgccttcgcgtcccttgtcagcccgtaccgccgacgtccgtggatgacggagcacggagcggcggctgcggacagcggccaggtgggccttaagggcatcgtcgccggcggtattacgggcggcatcgagatctgcatcacctatcccaccgagtatgtgaagacacaactgcagctggacgagaagggagcggcgaagaagtacaacggcatctttgattgcgttaagaagacggtgggagaacgtggattcctgggtctctaccgaggtctcagtgtgctcgtctacggcagcattcctaagtcagcggcgagattcggtgcttttgaattcctgaaatcgaacgccgtggactcccgaggacaactgagcaactccggaaagttgctttgcggcttaggagctggcgtatgtgaggccatcgtggcagtgaccccgatggagaccatcaaggtgaagttcatcaacgatcagcgcagcggcaatcccaagttcaggggattcgcccacggcgttggccaaatcatcaagtcggagggcatctctggtatctacaagggcctaacgcccaccattctgaaacagggctccaaccaggccattcgcttcttcgtactggagtcccttaaggatttgtacaagggtgacgatcacacgaagccagtgcccaagttggtggttggagtgtttggagctatcgccggagcagcctctgtgttcggtaatacgccgctggatgtggtcaagacgaggatgcagggtctggaagcatcgaaatacaagaacactgcccactgtgcggtggagatcttgaagaacgaaggtcccgccgccttctacaagggcaccgtgccaagattaggtcgtgtgtgcctggatgtggccatcacctttatgatctacgactccttcatggatctgttcaacaaggtgtgggtgtaactcggcgattcggcttggcgacttagttacttagttcatccttctaccccaaaaaacccactcgtgcattttcctctgttcccattgttccctagtcgactgttgcaatagtttctagttttgtgacatattccaagaaagcattttatatacactctatacccgcaatgggtaagtggctatagaaattatacagcttgttttaaaaattgaattaaatctgaagagtctatttgaagttcccatataaatcatagctagactggatgaaatag

SEQ ID NO: 12 - protein, 157 aa, Drosophila melanogaster
MSFVGKKYKLDKSENFDEYMKELGVGLVTRKMGNSLSPTVEVTLEGDTYTLTTTSTFKTSAISFKLGVEFDEETLDGRNVKSIITLDGNKLTQEQKGDKPTTIVREFTDNELITLIPHLTPSQHGSLRLRVPCQPVPPTSVDDGARSGGCGQRPGGP

SEQ ID NO: 13 - protein, 133 aa, Mus musculus (FABP 3)
MADAFVGTWKLVDSKNFDDYMKSLGVGFATRQVASMTKPTTIIEKNGDTITIKTQSTFKNTEINFQLGIEFDEVTADDRKVKSLVTLDGGKLIHVQKWNGQETTLTRELVDGKLILTLTHGSVVSTRTYEKEA

SEQ ID NO: 14 - protein, 135 aa, Mus musculus (FABP 5)
MASLKDLEGKWRLMESHGFEEYMKELGVGLALRKMAAMAKPDCIITCDGNNITVKTESTVKTTVFSCNLGEKFDETTADGRKTETVCTFQDGALVQHQQWDGKESTITRKLKDGKMIVECVMNNATCTRVYEKVQ

SEQ ID NO: 15 - protein, 132 aa, Mus musculus (FABP 7)
MVDAFCATWKLTDSQNFDEYMKALGVGFATRQVGNVTKPTVIISQEGGKVVIRTQCTFKNTEINFQLGEEFEETSIDDRNCKSVVRLDGDKLIHVQKWDGKETNCTREIKDGKMVVTLTFGDIVAVRCYEKA

SEQ ID NO: 16 - protein, 132 aa, Rattus norvegicus (FABP 7)
MVDAFCATWKLTDSQNFDEYMKALGVGFATRQVGNVTKPTVIISQEGGKVVIRTQCTFKNTEISFQLGEEFEETSIDDRNCKSVIRLDGDKLIHVQKWDGKETNCVREIKDGKMVVTLTFGDVVAVRCYEKA

SEQ ID NO: 17 - protein, 132 aa, Homo sapiens (FABP 7)
MVEAFCATWKLTNSQNFDEYMKALGVGFATRQVGNVTKPTVIISQEGDKVVIRTLSTFKNTEISFQLGEEFDETTADDRNCKSVVSLDGDKLVHIQKWDGKETNFVREIKDGKMVMTLTFGDVVAVRHYEKA

SEQ ID NO: 18 - protein, 132 aa, Drosophila melanogaster (FABP 7a)
MVDAFCATWKLVDSQNFDEYMKSLGVGFATRQVGNVTKPTIVISHEGDKVVIKTLSTFKNTEISFKLGEEFDETTADDRHVKSTVSLEGDNLVQVQRWDGKETKFVREIKDGKMVMTLTFEGVQAVRTYEKA

SEQ ID NO: 19 - protein, 130 aa, Drosophila melanogaster (CG31305PI); identical to SEQ ID NO: 10

SEQ ID NO: 20 - protein, 138 aa, Drosophila melanogaster (CG31305PJ)
MSFVGKKYKLDKSENFDEYMKELGVGLVTRKMGNSLSPTVEVTLEGDTYTLTTTSTFKTSAISFKLGVEFDEETLDGRNVKSIITLDGNKLTQEQKGDKPTTIVREFTDNELITLIPHLTPSQHGSLRLRVPCQPVPP

SEQ ID NO: 21 - protein, 117 aa, Drosophila melanogaster (CF31305PA); identical to SEQ ID NO: 8

(19) United States
(12) Patent Application Publication
(10) Pub. No.: US 2016/0051703 A1
(43) Pub. Date: Feb. 25, 2016

(54) METHODS AND COMPOSITIONS FOR IMPROVING SLEEP AND MEMORY

(71) Applicants: Charles F. Landry, Fitchburg, WI (US); Jerry C. Yin, Madison, WI (US); Jason R. Gerstner, Swarthmore, PA (US)

(72) Inventors: Charles F. Landry, Fitchburg, WI (US); Jerry C. Yin, Madison, WI (US); Jason R. Gerstner, Swarthmore, PA (US)

(21) Appl. No.: 14/860,143

(22) Filed: Sep. 21, 2015

Related U.S. Application Data
(63) Continuation of application No. 13/751,213, filed on Jan. 28, 2013.
(60) Provisional application No. 61/591,536, filed on Jan. 27, 2012.

(51) Int. Cl. A61K 49/00; G01N 33/50; C12N 15/113

(52) U.S. Cl. CPC A61K 49/0008; C12N 15/1138; G01N 33/5023; C12N 2310/14; C12N 2320/30; G01N 2333/705

(57) ABSTRACT

A method for determining whether a substance can increase the expression level of a fatty acid binding protein (FABP) in an animal. The method includes using a cell that includes an expression construct that comprises a FABP promoter operably linked to a polynucleotide sequence encoding a reporter molecule, wherein the cell is contacted with a candidate substance and then cultivating the cell under conditions conducive to expression of the reporter molecule. Increased expression of the construct in the presence of the candidate substance as compared to a control leads to improved sleep and long-term memory in the subject.

### CROSS-REFERENCE TO RELATED APPLICATIONS

This is a continuation of co-pending application Ser. No. 13/751,213, filed Jan. 29, 2013, which claims priority to provisional application Ser. No. 61/591,536, filed Jan. 27, 2012, which is incorporated herein by reference.

### FEDERAL FUNDING STATEMENT

This invention was made with United States government support awarded by the following agencies: National Institutes of Health, under the grant number MH67774, DA013780 and DA019513. The United States government has certain rights in this invention.

### BACKGROUND OF THE INVENTION

This invention relates to compositions and methods for improving sleep and memory in animals.

All animals require some form of sleep for survival. When deprived of sleep, animals display symptoms of sleep deprivation including lethargy, muddled thought processing, and loss of other cognitive abilities.

The sleep-wake cycle (e.g., the daily cycle typical for humans, and the polyphasic sleep-wake cycles in certain other animals) are regulated by a homeostatic mechanism and the circadian system. The two main regulated variables are sleep intensity and to a lesser extent, sleep duration (or the amount of sleep). Homeostasis refers to regulatory mechanisms that maintain physiological constancy of a living organism; when applied to sleep, it refers to the regulatory system that enables the organism to compensate for the loss of sleep or surplus sleep. In other words, the homeostatic mechanisms regulate sleep pressure or propensity. In contrast, the circadian clock regulates the timing of sleep.

The normal homeostatic sleep recovery after deprivation (“rebound”) is one of the two fundamental qualities that define sleep in animals. All animals show rebound after sleep deprivation. Amount of sleep needed to recover from sleep deprivation depends on the animal and the quality of their sleep.

Sleep and memory are generally believed to be linked. Many researchers believe that the mind processes experiences from throughout the day and stores them as memories during sleep.

While sleep is known to be critical for survival, the biological and physiological mechanisms of sleep and memory processing are not understood. In order to start to understand the biochemistry of sleep, researchers have explored the gene expression patterns in brains of animals during sleep. A number of genes have been identified as candidates involved in sleep regulation and sleep processes.

At present, however, there is no known gene whose expression level can be manipulated to improve sleep and memory.

### SUMMARY OF THE INVENTION

The present inventors discovered that increased expression levels of fatty acid binding protein (FABP) leads to improved sleep quality and improved long-term memory in animals.

Accordingly, the present disclosure describes assays for identifying compounds that increase the expression level of a FABP gene in a cell. The disclosure also describes a method comprises increasing the expression of an endogenous FABP gene, whereby sleep quality and/or long-term memory are improved. The assay comprises contacting a test cell which comprises an expression vector comprising a suitable FABP promoter operably linked to a suitable reporter sequence, with a test compound under suitable conditions for a time sufficient to allow the test compound to increase the FABP expression level in the test cell. An increase in the FABP expression level in the test cell contacted with the test compound relative to a test cell not contacted with the test compound indicates that the test compound increases the expression activity of FABP in a cell comprising an endogenous FABP gene.

Test compounds selected using the above-noted primary assay can be used as lead compounds for the development of therapeutic agents useful for improving sleep quality and long-term memory.

Also described herein are in vivo and other secondary assays to further characterize the test compounds identified using the primary assay. Specifically, the secondary assay provides transgenic animals, such as transgenic fruit flies, that comprise the expression vector described above, which transgenic animals can be used to determine the test compounds' ability to increase FABP expression in vivo.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, 5, 6, from 1 to 9, from 3.6 to 4.6, from 3.5 to 9.9, and so forth.

All references to singular characteristics or limitations of the present invention shall include the corresponding plural characteristic or limitation, and vice-versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

The methods of the present invention can comprise, consist of, or consist essentially of the essential elements and limitations of the method described herein, as well as any additional or optional ingredients, components, or limitations described herein or otherwise useful in genetics, microbiology, physiology, and/or chemistry.

Other objects, advantages and novel features of the present invention will become apparent from the following detailed description when considered in conjunction with the accompanying figures. An individual skilled in the art will understand that the descriptions below are for illustration purposes only, and are not intended to limit the scope of the present teachings in any way.

### BRIEF DESCRIPTION OF THE DRAWINGS

FIG. 1 provides the nucleotide sequences of the human and mouse FABP3, FABP5 and FABP7 genes, including their promoter regions. (Human FABP3=SEQ. ID. NO: 1; Human FABP5=SEQ. ID. NO: 2. Human FABP7=SEQ. ID. NO: 3. Murine FABP3=SEQ. ID. NO: 4. Murine FABP5=SEQ. ID. NO: 5. Murine FABP7=SEQ. ID. NO: 6.)

FIG. 2A shows a comparison of the amino acids in various mammalian FABP7 genes. FIG. 2B depicts the structure of the Drosophila FABP gene (dFABP). Drosophila melanogaster fatty acid binding protein, transcript variant A, mRNA 1,042 bp, mRNA Accession: NM_{—}001032010.1 (SEQ. ID. NO: 7; CDS=SEQ. ID. NO: 8). Drosophila melanogaster fatty acid binding protein, transcript variant B, mRNA 714 bp, mRNA Accession: NM_{—}001032009.1 (SEQ. ID. NO: 9; CDS=SEQ. ID. NO: 10). Drosophila melanogaster fatty acid binding protein, transcript variant C, mRNA 1,661 bp, mRNA Accession: NM_{—}001032008.1 (SEQ. ID. NO: 9). Unless a variant is specifically noted, variants A, B, and C are collectively referred to as “dFABP.”

FIGS. 3A, 3B and 3C compare the baseline sleep profiles for the three mouse strains under constant conditions (strains 2U, 103-3, and 101-4). In each of FIGS. 3A, 3B, and 3C, the X-axis is time in hours, the Y-axis is time asleep in minutes. FIGS. 3D, 3E, 3F, 3H, 3I, and 3J provide quantitative analyses thereof. FIG. 3D is a histogram depicting total sleep in minutes over the 24-hour test period. FIG. 3E depicts the average time asleep in minutes (bout length) for each period of slumber during daylight hours for each strain. FIG. 3F depicts the maximum bout length in minutes for periods of slumber during daylight hours for each strain. FIG. 3H depicts the number of bouts of sleeping during the 24-hour test period for each strain tested. FIG. 3I depicts the average time asleep in minutes (bout length) for each period of slumber during nighttime hours for each strain. FIG. 3J depicts the maximum bout length in minutes for periods of slumber during nighttime hours for each strain.

FIGS. 4A, 4B, 4C, 4D, 4E, 4F, 4G, and 4H taken together provide an analysis of two additional independent insertions of the dFABP transgene (101-2 and 101-6). FIG. 4A depicts bouts of sleep (in minutes) over the 24-hour study period for strain 2U. FIG. 4B depicts bouts of sleep (in minutes) over the 24-hour study period for strain 101-2. FIG. 4C depicts bouts of sleep (in minutes) over the 24-hour study period for strain 101-4. FIG. 4D depicts bouts of sleep (in minutes) over the 24-hour study period for strain 101-6. FIG. 4E depicts total sleep time for all four strains over the 24-hour test period. FIG. 4F depicts daytime sleep (in minutes) during the test period. FIG. 4G depicts the number of bouts of sleep over the 24-hour test period. FIG. 4H depicts the average time (in minutes) of daytime bouts of sleep.

FIGS. 5A, 5B, 5C, 5D, 5E, 5F, 5G, and 5H taken together, show that mild overexpression of FABP7 or dFABP specifically enhances long-term memory (LTM). FIG. 5A depicts performance index as a function of temperature for strains 103-3 and 2U. FIG. 5B depicts performance index ratio as a function of temperature. FIG. 5C depicts performance index (1X) for FABP7 overexpression in strain 103-3. FIG. 5D depicts performance index (1X) for dFABP overexpression in strain 101-4. FIG. 5E depicts performance index (1X) for dFABP overexpression in strain 101-9. FIG. 5F depicts performance index (10X) for FABP7 overexpression in strain 103-3. FIG. 5G depicts performance index (10X) for dFABP overexpression in strain 101-4. FIG. 5H depicts performance index (10X) for dFABP overexpression in strain 101-9.

FIG. 6 is a gel depicting the increase in dFABP protein as a function of temperature in both nuclear and cytoplasmic compartments from head extracts.

FIG. 7A plots the percentage change in sleep (trained/untrained) for the three different genotypes that were tested: 2U, 103-3, and 101-4. FIGS. 7B, 7C, and 7D present the quantitation of the changes on an hour-by-hour basis for the three different genotypes, respectively. FIG. 7B shows the results for strain 101-4. FIG. 7C shows the results for strain 103-3. FIG. 7D shows the results for strain 2U.

FIG. 8 is a graph showing that the FABP transgenic flies have greater resistance to chronic sleep deprivation.

FIG. 9 is a graph showing that the dFABP transgenic flies exhibit faster recovery from anoxia.

FIG. 10 is a graph showing that dFABP transgenic flies are more resistant to hyperoxia.

### DETAILED DESCRIPTION OF THE INVENTION

Many genes involved in neurobiological processes are highly conserved in structure and function, and are similarly regulated in Drosophila as in mammals. As a consequence, Drosophila has long served as a model system for mammalian neurobiological research, including in the areas of circadian biology, memory and sleep (Yin, J. C., et al., Cell, 1994. 79: p. 49-58; Yin, J. C., et al., Cell, 1995. 81: p. 107-15; Josselyn, S. A., et al., J Neurosci, 2001. 21: p. 2404-12), anesthesiology (Humphrey, J. A., et al., Curr Biol, 2007. 17: p. 624-9), aggression (Chen, S., et al., Proc Natl Acad Sci USA, 2002. 99: p. 5664-8), ethanol (Guarnieri, D. J. and U. Heberlein, Int Rev Neurobiol, 2003. 54: p. 199-228), and other substance addictions (Wolf, F. W. and U. Heberlein, J Neurobiol, 2003. 54: p. 161-78).

All animals contain proteins that bind fatty acids. All fatty acid binding proteins (FABPs) are members of a large multigene family now called “intracellular lipid binding proteins” (iLBPs). Despite the considerable differences in their primary structure, the tertiary structure of all iLBPs is highly conserved. A total of eight FABP-types are expressed in various mammalian tissues each carrying out distinct metabolic tasks. One type, B-FABP, expressed in mammalian brain and neurons, is especially conserved (see e.g. Haunerland and Spener, 2004, Adv. Mol. Cell Biol. 33: 99-123). The human B-FABP protein (as well as the gene encoding it) is also known as FABP-7.

The present inventors discovered that increased levels of expression of one or more FABPs especially B-FABP, and most preferably FABP-7, leads to improved sleep quality, improved long-term memory, and improved cognition in animals. Accordingly, described herein is a method for improving sleep quality, long-term memory, and cognition by increasing the expression of one or more genes that encode at least one FABP. Also described are assays for identifying compounds that increase the expression level of a FABP gene in a cell which comprises an endogenous FABP gene. Such compounds are useful for improving sleep quality and/or for improving long-term memory and/or cognition in an animal, including humans.

The method described herein preferably is a cell-based method, wherein a test cell is genetically engineered to comprise an expression vector that contains a suitable regulatory element, such as a promoter, that controls or regulates the expression of a FABP gene, operably-linked to a suitable reporter sequence. In one version of the method, the test cell is contacted with a candidate or test compound under suitable conditions for a sufficient time to allow the test compound to exert its effect. If the test compound is found to increase the expression level of the reporter sequence under the control of the FABP regulatory sequence in the test cell, as compared to a suitable control wherein the test cell is not contacted with the test compound, the test compound would be considered to have the ability to increase the expression activity of FABP in a cell which comprises an endogenous FABP gene, including such cells in vivo, and may be chosen for further testing, such as testing using a suitable animal model.

Test compounds selected from the above primary assays are useful as lead compounds for the development of therapeutic agents for improving sleep quality and long term memory.

Thus, in another version of the method, an in vivo secondary assay is described and which can be used to further characterize the candidate compounds. For example, a suitable amount of the test compound, in a suitable formulation, may be administered to a test animal, and its effect on sleep and memory of the animal studied.

FABP Promoters/Expression Regulatory Elements:

Promoters or other cis-acting elements that regulate the expression of FABP genes are known in the art. For purposes of this disclosure, a cis-element or cis-acting element is a region of DNA or RNA that regulates the expression of one or more genes located on that same molecule of DNA. In this sense, cis-elements are “operationally linked” to the genes they control, even though they may not be directly bonded to the coding sequence. That is a cis-element operationally linked to a gene encoding a protein has a regulatory effect on the expression of the gene, even if the cis-element is far removed from the gene. A person of ordinary skills in the art will readily be able to obtain the promoter for any FABP gene based on publicly or commercially available materials and information. For example, a wide range of promoters and reporters that will function in various eukaryotic cells are available commercially from, for example, Lucigen (Middleton, Wis.) Promega (Madison, Wis.), and Life Technologies (Grand Island, N.Y.). (Life Technologies is a conglomerate that wholly owns several companies that supply promoters and reporters, including Invitrogen, Applied BioSystems, Gibco, Novex, Molecular Probes, TaqMan, and Ambion.)

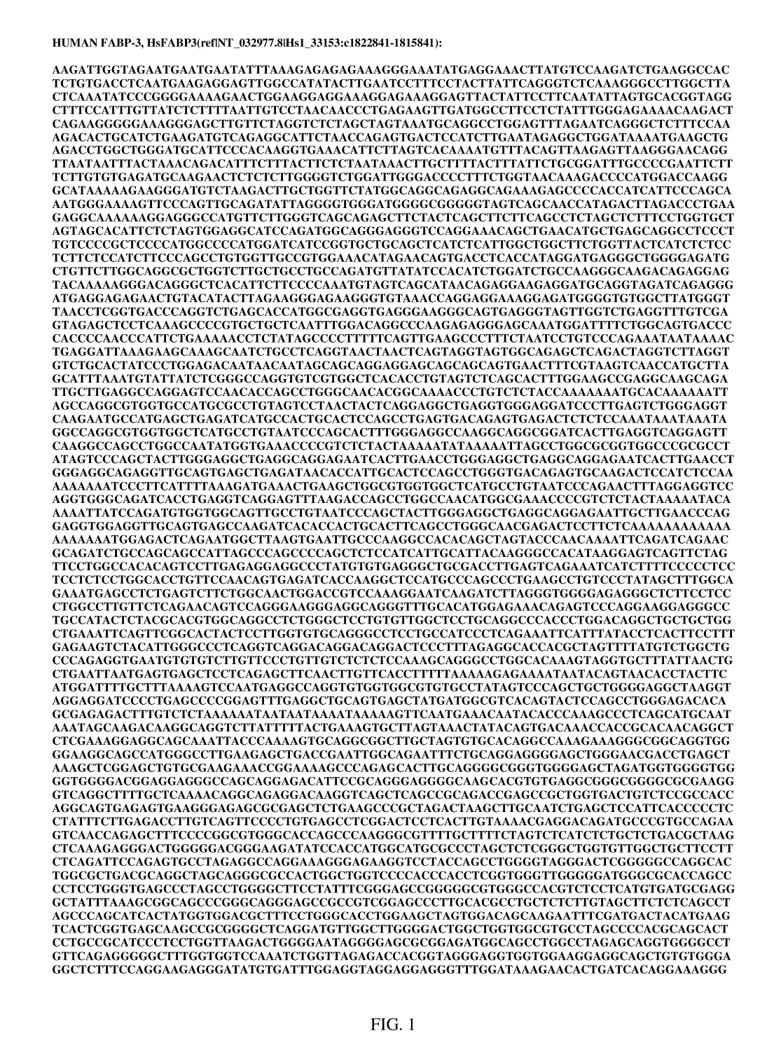

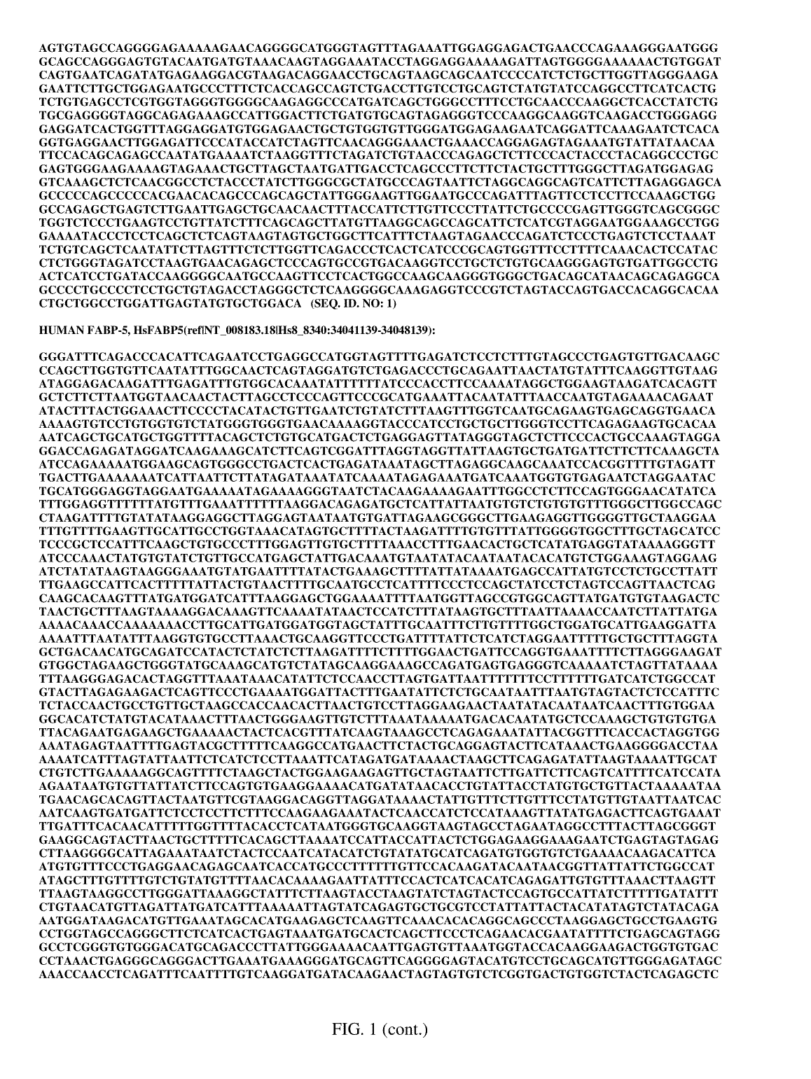

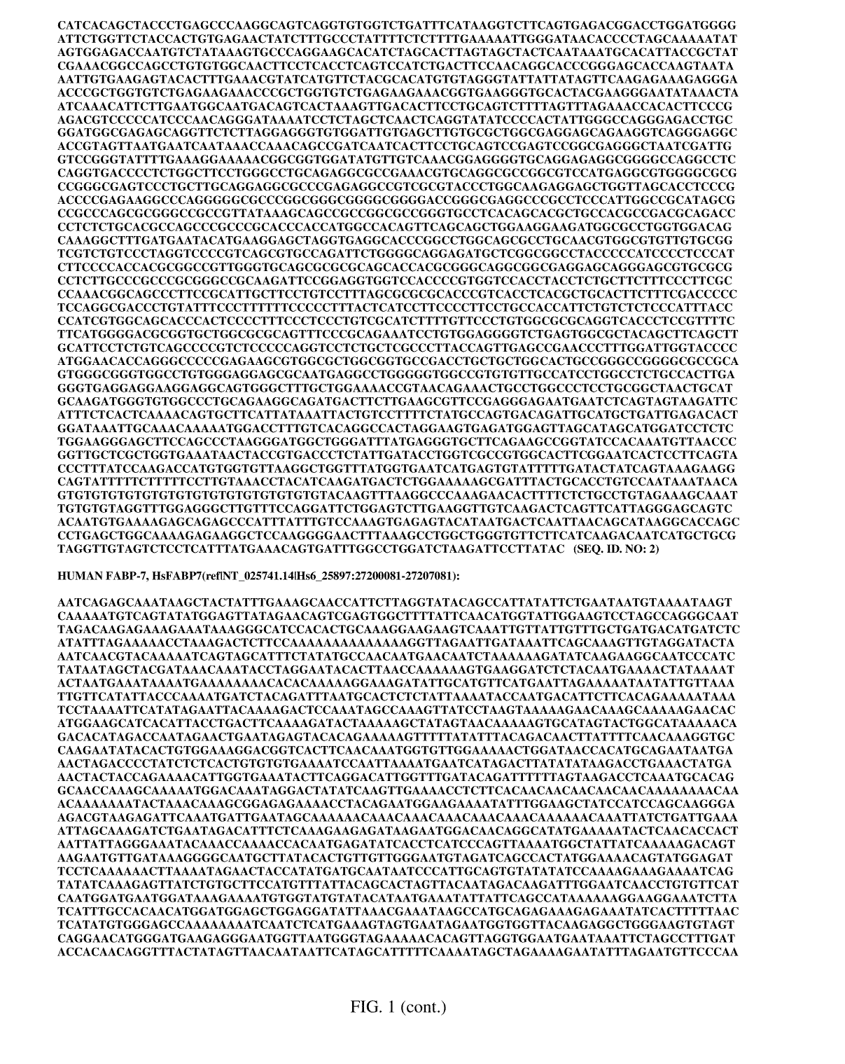

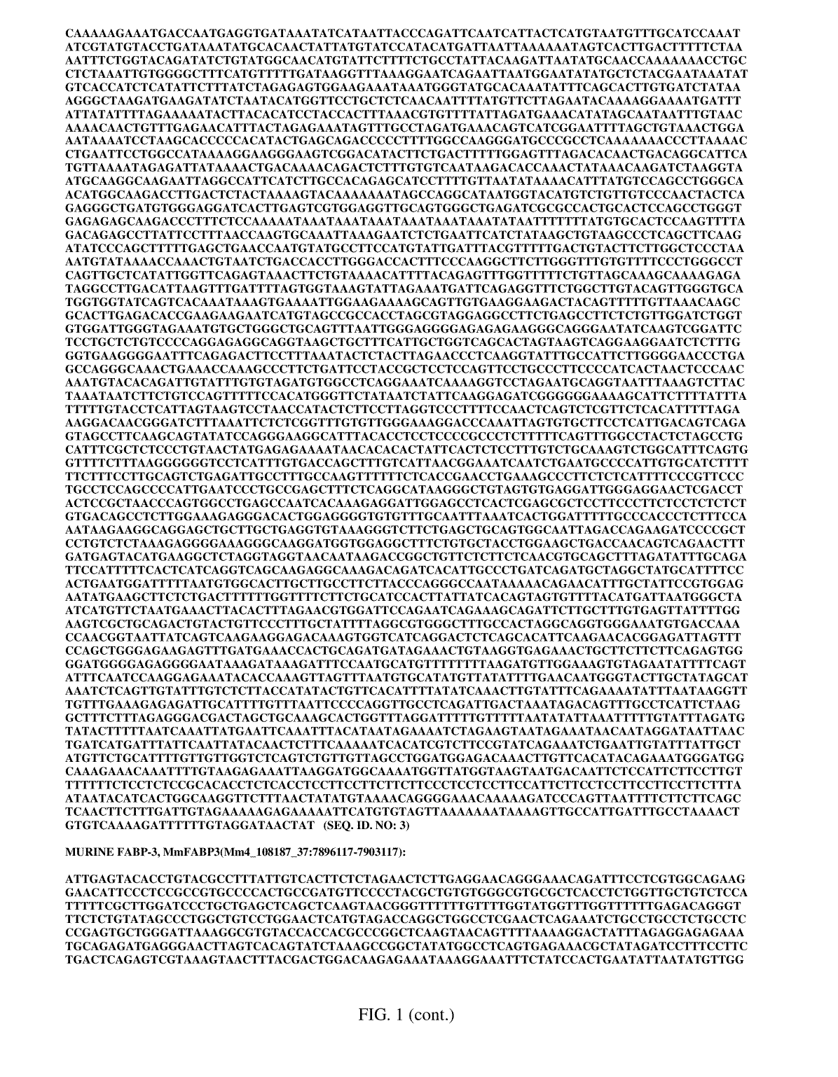

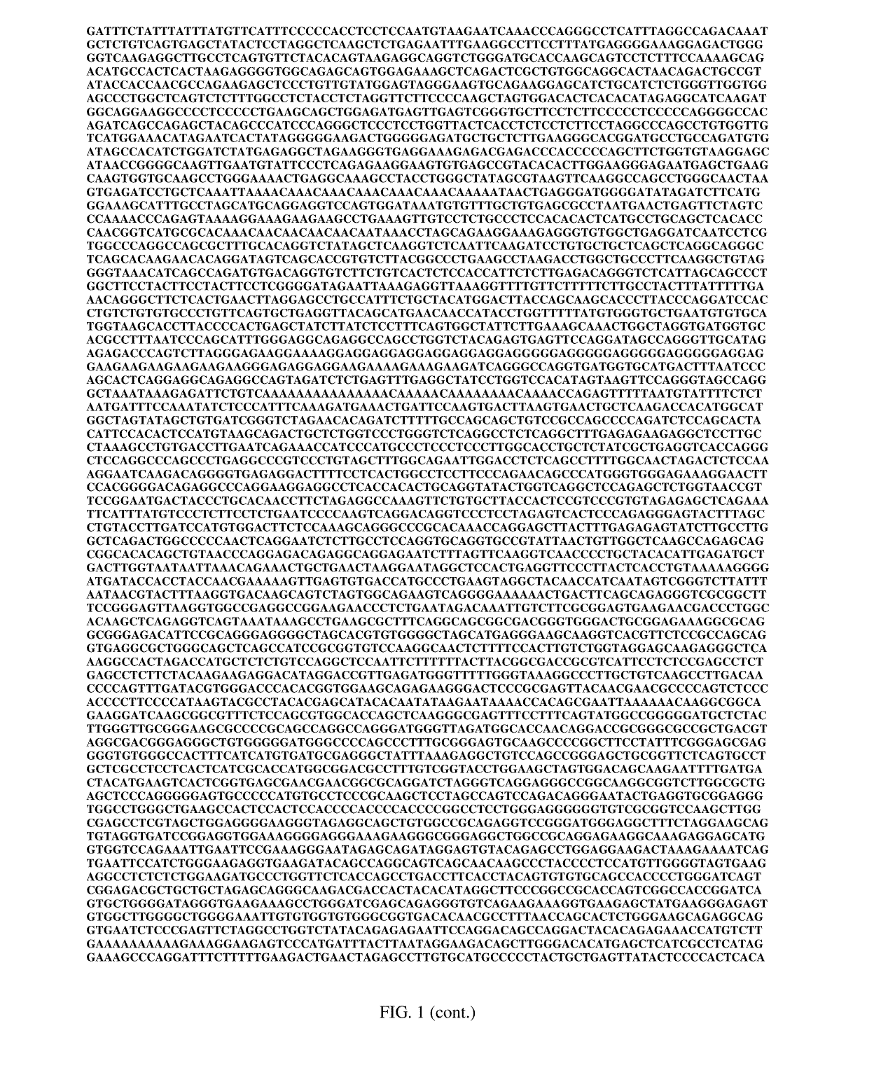

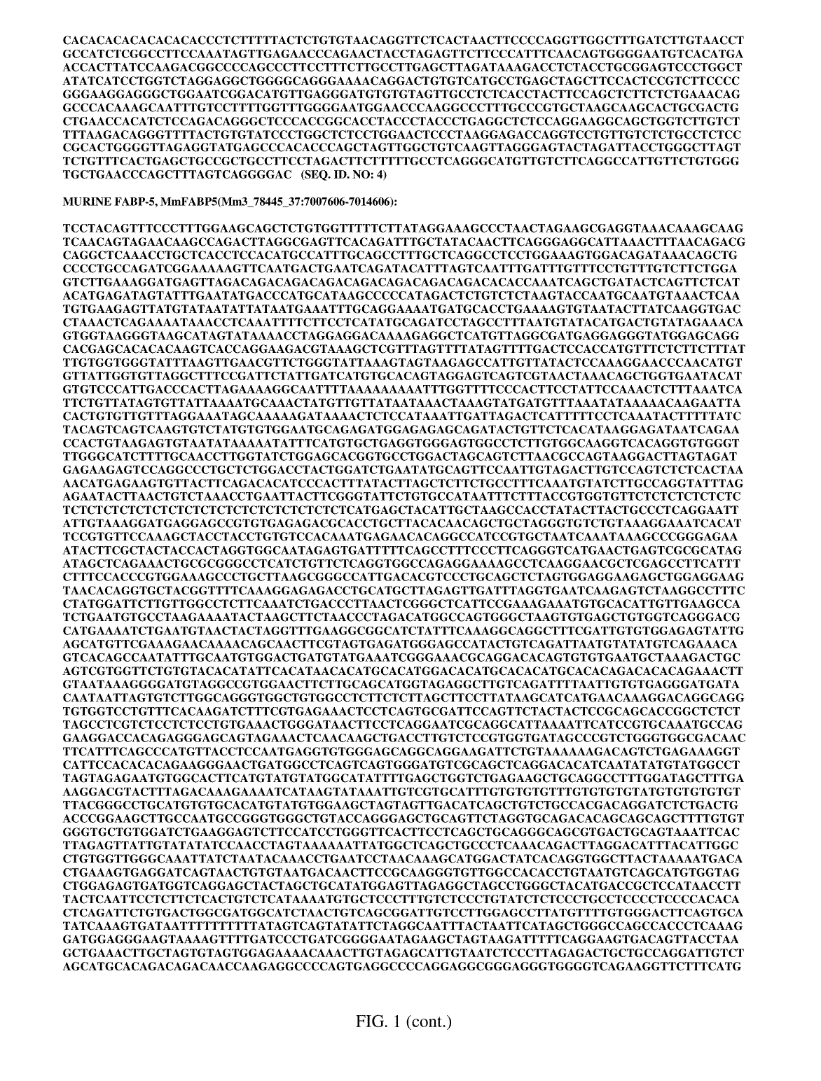

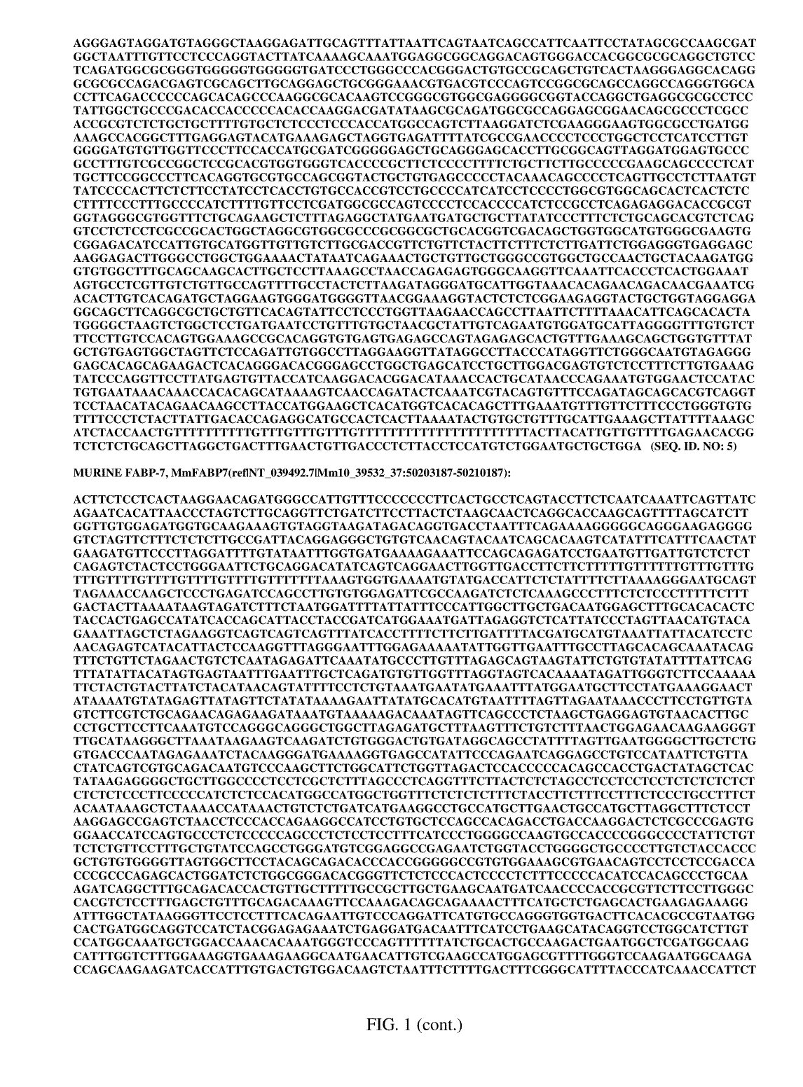

FIG. 1 provides the nucleotide sequences of the promoters of the human and mouse FABP3, FABP5 and FABP7 genes. See also SEQ. ID. NOS: 1-6, respectively. These three genes are known to be expressed in adult mammalian central nervous systems. Driving overexpression of one or more of the gene products of these genes has been shown by the present inventors to improve sleep, cognition, and long-term memory in accepted animal tests. (See the examples.)

An example of a minimum FABP promoter is described in Feng and Heintz, 1995, Development 121:1719-1730, and in Anthony et al., 2004, Neuron 41:881-890). Other FABP promoter regions are described in Schachtrup et al., Functional analysis of peroxisome-proliferator-responsive element motifs in genes of fatty acid-binding proteins, Biochem. J. (2004) 382, 239-245; (L-FABP) and (A-FABP); Issemann et al., (1992) A role for fatty acids and liver fatty acid binding protein in peroxisome proliferation? Biochem. Soc. Trans. 20, 824-827; Simon et al., (1993) Use of transgenic mice to map cis-acting elements in the liver fatty acid-binding protein gene (Fabpl) that regulate its cell lineage-specific, differentiation-dependent, and spatial patterns of expression in the gut epithelium and in the liver acinus. J. Biol. Chem. 268, 18345-18358; Tontonoz (1994), Adipocyte-specific transcription factor ARF6 is a heterodimeric complex of two nuclear hormone receptors, PPARγ and RXRα. Nucleic Acids Res. 22, 5628-5634; Wolfrum et al. (1999) Phytanic acid is ligand and transcriptional activator of murine liver fatty acid binding protein. J. Lipid Res. 40, 708-714; Pelton et al., (1999) PPARγ activation induces the expression of the adipocyte fatty acid binding protein gene in human monocytes. Biochem. Biophys. Res. Commun. 261, 456-458; Bleck et al., (1998) Cloning and chromosomal localisation of the murine epidermal-type fatty acid binding protein gene (Fabpe). Gene 215, 123-130, and Treuner et al., (1994) Cloning and characterization of the mouse gene encoding mammary-derived growth inhibitor/heart-fatty acid-binding protein. Gene 147,237-242. All of these references are incorporated herein in their entirety.

In particular, Bleck et al. (1998) describes the murine promoters of E-FABP gene (Fabpe), and Truener et al. (1994) describes the H-FABP promoter, both of which may serve as templates to generate promoter fragments.

Reporter Genes:

The term “reporter gene” is defined to mean any genetic sequence that is detectable and distinguishable from other genetic sequences present in test cells. A suitable reporter molecule allows the expression level of the FABP promoter to be assayed. Preferably, the reporter gene sequence encodes a protein that is readily detectable either by its presence, or by its activity that results in the generation of a detectable signal. A reporter gene can be used in the invention to monitor and report the expression level of the FABP promoter in the presence or absence of a candidate substance. A very large number of reporter genes are available from the commercial sources listed previously for promoters.

Commonly used reporter genes that induce visually identifiable characteristics usually involve fluorescent proteins; examples include the gene that encodes jellyfish green fluorescent protein (GFP), which causes cells that express it to glow green under UV light, and the enzyme luciferase, which catalyzes a reaction with a luciferin to produce light. (Both of these reporters are available commercially from Promega.) Other common reporter genes in bacteria include the lacZ gene, which encodes the protein β-galactosidase, an enzyme that causes bacteria expressing the gene to appear blue when grown on a medium that contains the substrate analog X-gal (an inducer molecule such as IPTG is also needed under the native promoter). An example of a selectable-marker reporter in bacteria is the chloramphenicol acetyltransferase (CAT) gene, which confers resistance to the antibiotic chloramphenicol. Another example is the gene that encodes GUS (beta-glucuronidase).

Recombinant Gene Product:

In the present invention, a promoter for a FABP gene is engineered into an expression vector, which comprises a FABP promoter sequence operably linked to a nucleic acid sequence encoding a reporter molecule.

Techniques and materials needed for the construction of expression vectors are well established and well-known to those skilled in the art, including virus or plasmid based vectors. The terms “operably associated” or “operably linked,” as noted above, refer to an association in which a promoter and a nucleic acid sequence to be expressed are linked (directly or indirectly) and positioned in such a way as to permit transcription of the coding sequence that is operably linked to the promoter. Two or more sequences, such as a promoter and any other nucleic acid sequences are operably-associated if transcription commencing in the promoter will produce an RNA transcript of the operably-associated sequence(s). A suitable expression vector useful in the invention may also contain selectable or screenable marker genes for initially isolating, identifying or tracking recombinant cells that contain the vector.

Host Cells:

Depending on the specifics of the expression construct, many suitable cells can be used as the host cell for the expression vector, including bacterial, yeast, insect, and mammalian cells originated from various tissues, including central nervous system cells. For example, C6 Glioma cells are commercially available from Amaxa Inc., Gaithersburg, Md.). These are well-known and readily available to those skilled in the art.

Small Molecule Libraries:

Agents to be screened can be naturally occurring or synthetic molecules, e.g., substances isolated from natural sources such as marine microorganisms, algae, plants, fungi, etc. Alternatively, agent to be screened can be from combinatorial libraries of agents, including peptides or small molecules, or from existing repertories of chemical compounds known in the art.

Combinatorial libraries can be produced for many types of compounds. Such compounds include polypeptides, proteins, nucleic acids, beta-turn mimetics, polysaccharides, phospholipids, hormones, steroids, aromatic compounds, and heterocyclic compounds. Many large combinatorial libraries of compounds are known or can be constructed by methods well-known to those skilled in the art (see e.g. WO 95/12608, WO 93/06121, WO 94/08051, WO 95/35503, WO 95/30642, and WO 91/18980 (each of which is incorporated herein by reference in its entirety). For example, many compounds to be screened can also be obtained from governmental or private sources, including, e.g., the National Cancer Institute's (NCI) Natural Product Repository, Bethesda, Md., the NCI Open Synthetic Compound Collection, Bethesda, Md., NCI's Developmental Therapeutics Program, and the like.

Screening may be based on morphology or motility of cells, or on the transcriptional readouts after enhanced expression of the reporter sequence (e.g. via microarray-based assays). Screening may also be an ELISA-based screen (see e.g. Cho et al., A simple method to screen ligands of peroxisome proliferator-activated receptor delta. Eur J Pharm Sci. 2006, 29:355-60).

In vivo Tests:

To further characterize the test compounds, the compounds may be administered to a test animal, and its effect on sleep and memory of the animal studied. For example, a dicistronic expression vector may be engineered into a suitable host cell, wherein the vector comprises a FABP promoter operably linked to a FABP ORF and a reporter gene (e.g. the luciferase gene, or other reporter, as noted above). For an illustration of a similar construct, see e.g. Belvin et al., 1999, The Drosophila dCREB2 gene affects the circadian clock. Neuron. 1999 22:777-87.). A test compound can be administered to a transgenic animal comprising such a construct and its effect tested in vivo.

It is well known in the art that animal behavioral criteria can be used to measure the effect of test compounds. For example, Shaw, 2003, Awakening to the behavioral analysis of sleep in Drosophila. J Biol Rhythms. 2003 February; 18(1):4-11, discloses the use of fruit fly sleep behavior to test drug efficacy, and Drier et al., 2002, Nat Neurosci. 2002 April; 5 (4):316-24, discloses a method using fly memory behavior. Similarly, compounds can be screened against sleep deprivation-induced memory impairments (Ganguly-Fitzgerald et al., 2006, Science. 2006 September 22; 313(5794):1775-81.)

Similar methodologies are also established and known in the mouse model. See e.g. Anthony et al., 2005, Genes Dev. 2005 May 1; 19(9):1028-33; Schmid et al., 2006, Glia. March; 53(4):345-51; Tang and Sanford, 2002, Sleep 25:691-699; and McDermott et al., 2003, J Neurosci. 2003 October 22; 23(29):9687-95. As it is known that sleep deprivation following training inhibits memory in flies (Ganguly-Fitzgerald et al., 2006, supra) and mice (McDermott et al., 2003, supra), a compound may be tested to see if it can attenuate memory impairments following sleep deprivation.

In another embodiment, the present disclosure provides a method for improving sleep quality or memory, especially long-term memory, in an animal in need thereof, the method comprising administering to the animal an effective amount of the pharmaceutical composition comprising a suitable FABP agonist and a pharmaceutically acceptable carrier.

The present disclosure also provides a method for increasing sleep time in an animal in need thereof, the method comprising administering an effective amount of FABP antagonists, such as an anti-FABP encoding antisense nucleotide molecule, or a suitable siRNA molecule, or an anti-FABP antibody.

Aspects of the present teachings may be further understood in light of the following examples, which should not be construed as limiting the scope of the attached claims in any way.

### EXAMPLES

### Example 1

Experimental Details:

Mouse cDNA against the Fabp7 open reading frame was cloned into a heat shock vector as reported previously (Drier E A, Tello M K, Cowan M, Wu P, Blace N, Sacktor T C, Yin J C. Memory enhancement and formation by atypical PKM activity in Drosophila melanogaster. Nat Neurosci. 2002 April; 5(4):316-24.), and microinjected into fruit fly (Drosophila melanogaster) embryos for the generation of transgenic flies which can express mouse Fabp7 (103-3; Best Gene, Chino Hills, Calif.).

Olfactory Conditioning Assay: Drosophila learning and memory are tested using the olfactory-avoidance classical (Pavlovian) conditioning protocol, with automated and repetitive training regimens using 3-octanol (OCT) and 4-methylcyclohexanol (MCH) odors. Detailed descriptions of single-cycle, massed and spaced training, as well as testing, and the tests for olfactory acuity and shock reactivity are performed according to Drier et al. (2002). Performance is calculated by subtracting the number of flies making the incorrect choice from those making the correct one, and dividing the total number of flies in each experiment. To avoid odor-avoidance biases, an average performance of two groups of flies (one having the conditioned stimulus (CS+) being OCT, and the other CS+ being MCH) is calculated. Differences in learning and memory performance between groups is then tested by ANOVA, and post-hoc analysis (t-test) is used to examine the degree of significance (α=0.05).

Sleep in 103-3 flies was assayed using the Drosophila Activity Monitoring System (TriKinetics, Waltham, Mass.) as described previously (Andretic R, Shaw P J. Essentials of sleep recordings in Drosophila: moving beyond sleep time. Methods Enzymol. 2005; 393:759-72.). Briefly, flies were recorded for infrared beam-breaks to monitor their activity. These beam-breaks were recorded over 1-minute bins and averaged for every hour. Sleep deprivation was done using the automated sleep nullifying apparatus (SNAP, Shaw, P J, Tononi, G, Greenspan, R J, and Robinson, D F. (2002). Stress response genes protect against lethal effects of sleep deprivation in Drosophila. Nature 417(6886), 287-291), and recovery sleep was measured.

Comparison and Characterization of dFABP

FIG. 2A shows a comparison of the amino acids in various mammalian FABP7 genes. The mouse (mm), rat (Rn), human (Hs), and zebrafish (Dr) proteins are compared with the unique Drosophila FABP proteins (CG31305 PA, PI and PJ, which have been renamed CG6783-RA, RB and RC). The fly FABP gene, dFABP, is most similar to mammalian FABP7, and less similar to the other mammalian FABPs that are expressed in the nervous system (mFABP3 and mFABP5). FABP7 is more similar to dFABP than it is to any other mammalian FABP (data not shown). These comparisons suggest that mFABP7 and dFABP are closely related, and may be able to substitute functionally for each other.

FIG. 2B depicts the structure of the dFABP gene. The gene contains 2 different ORFs (RA/RC and a mitochondrial protein shown in the stippled box) whose translation is out-of-frame relative to each other. Exons are shown in boxes, and introns depicted with horizontal lines. The ORF for CG6783-RB (dFABP) is shown in green boxes, and its 5′ and 3′ UTR sequences are shown in gray boxes. The alternative splicing that produces CG6783-RB is shown in black above the exons. The translation start codon is indicated as ATG, and the termination codon is labeled STOP. CG6783-RA is an alternatively spliced form of this gene, and its splice sites are indicated with red lines. The inclusion of the red exon terminates translation that begins at ATG. ATG-2, shown in gray, is thus most likely to be used to make a protein that has unique amino acids at its N-terminus, which are added to the C-terminal parts of the ORF. The function of this predicted protein is unknown. The third alternatively spliced isoform is CG6783-RC, whose splicing pattern is shown in dotted black lines. The black stippled ORF codes for a mitochondrial carrier protein. The 3′ UTR of this ORF is not shown. The location of the single known P-element insert (G88) in the intron is shown with a blue triangle. All the experiments described in the instant application are done with the CG6783-RB isoform.

Modest Over-Expression of FABP7 or dFABP Decreases Daytime Sleep:

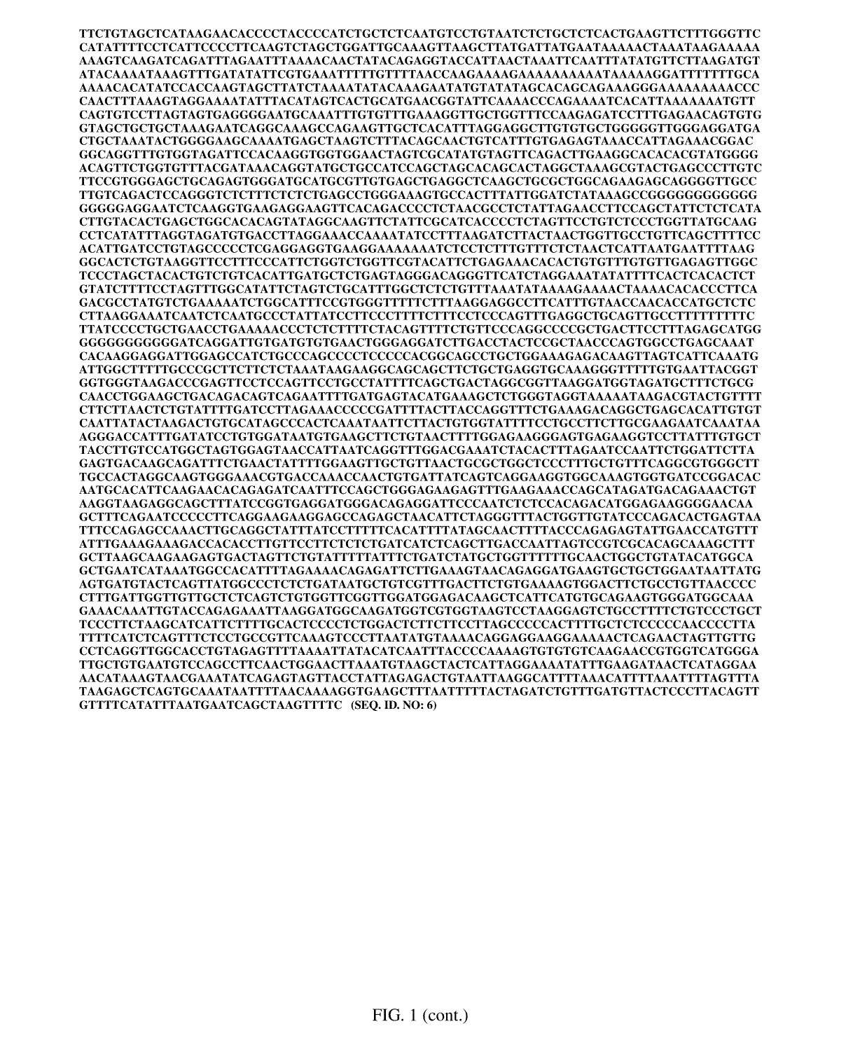

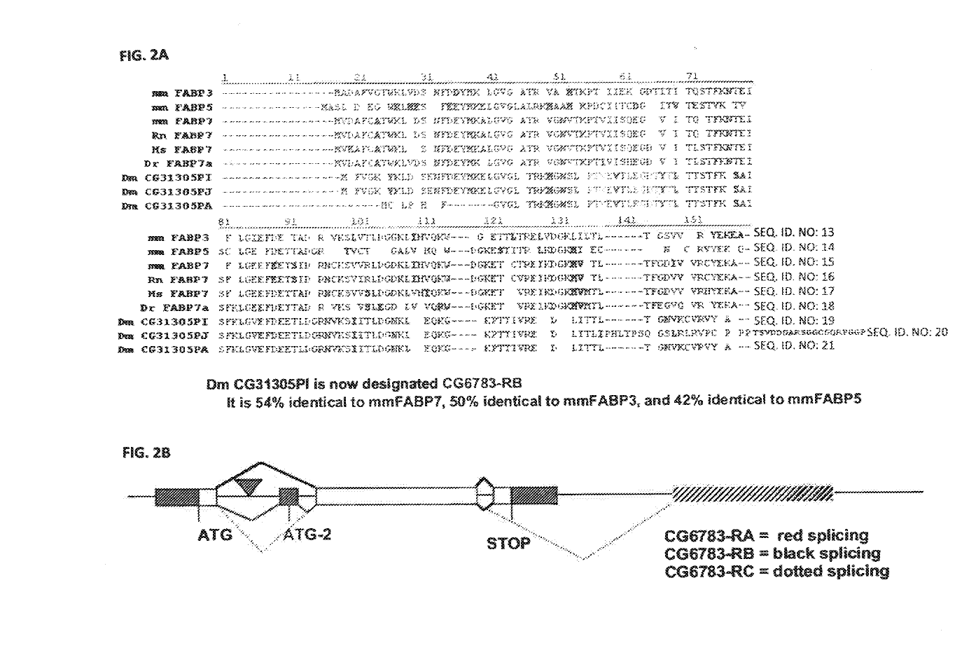

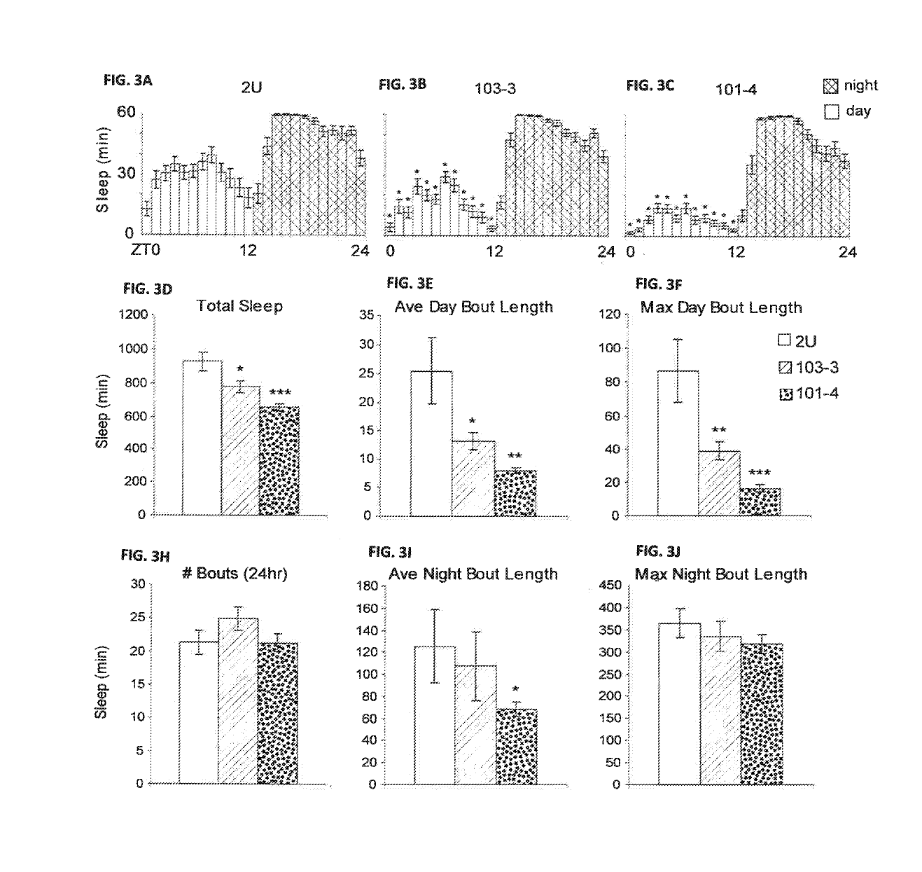

Using heat-shock promoter-driven transgenic flies, we have tested the effects of moderate overexpression of FABP7 (103-3) or dFABP (101-4) on steady-state sleep. The transgenic flies were made in the wild type background strain (2U), which serves as the isogenic control for all transgenic flies. The X-axis in FIGS. 3A, 3B, and 3C represents the 24 one-hour bins across the 12-hour light/12-hour dark circadian light regimen. The lights go on at ZT=0, and go off at ZT=12. The Y-axis shows the average amount of sleep during each of the 1-hour bins. Measurements are SEM. N=23-24, * p<0.05, ** p<0.01, *** p<0.001, t-test.

FIGS. 3A, 3B, and 3C compare the baseline sleep profiles for the three strains under constant conditions. The white histogram represents 2U, while the light (103-3) and dark gray (101-4) histograms represent the transgenic lines. All of the data were collected at room temperature (˜23° C.).

The most obvious effects are on the amount of daytime sleep. A quantitative analysis is presented in FIGS. 3D, 3E, 3F, 3H, 3I, and 3J. While the total amount of sleep across the entire day is decreased for both transgenic lines when compared to 2U, the bulk of this effect is due to effects on daytime sleep. The daytime sleep is considerably less “consolidated,” since both the average daytime bout length (FIG. 3E) and the maximum daytime bout length are significantly decreased. There are some subtle effects on nighttime sleep parameters in the 101-4 line (FIG. 3I, average length of nighttime bouts).

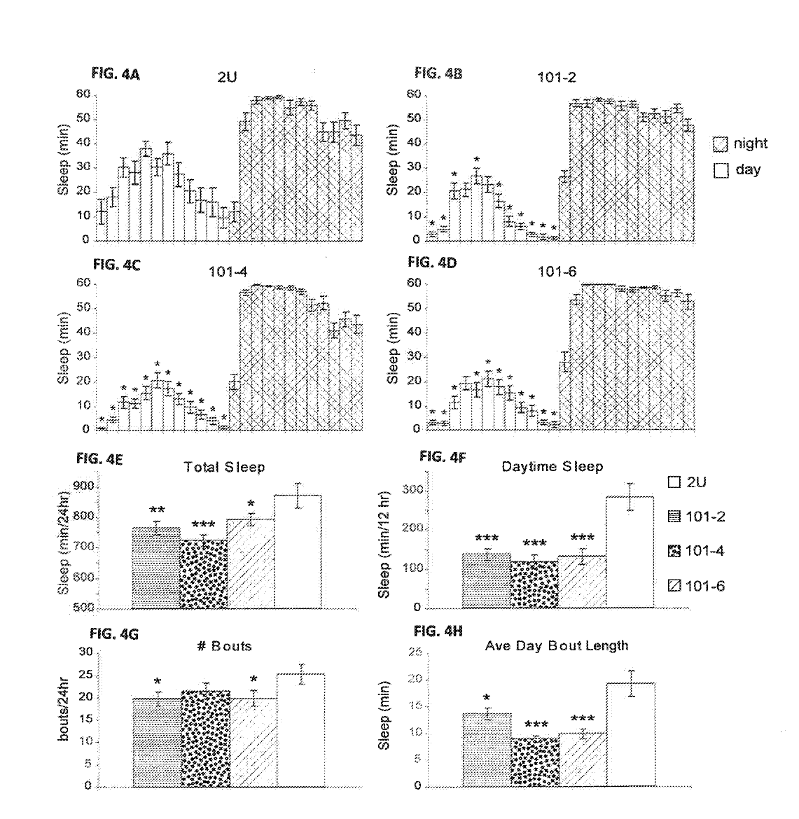

The generality of this finding is supported through the analysis of two additional independent insertions of the dFABP transgene (101-2 and 101-6). These two lines are compared to 2U and 101-4 in FIGS. 4A-4H. Measurements are SEM. N=22-24, * p<0.05, ** p<0.01, *** p<0.001, t-test.

FIGS. 4A, 4B, 4C, and 4D are the sleep profiles for the four different fly lines. FIGS. 4E, 4F, 4G, and 4H show more detailed analyses of various parameters. Again, alterations in daytime sleep are the largest effects, both in terms of total time spent sleeping during the daytime (FIG. 4F), which is responsible for the bulk of the decreased sleep across the entire 24 hour day/night (FIG. 4E). Together with the data in FIGS. 3A-3J, we can conclude that either FABP7 or dFABP overexpression affect daytime sleep. Since multiple insertions show the same effects, the results are independent of insertion sites.

Mild Overexpression of FABP7 or dFABP Specifically Enhances Long-Term Memory (LTM):

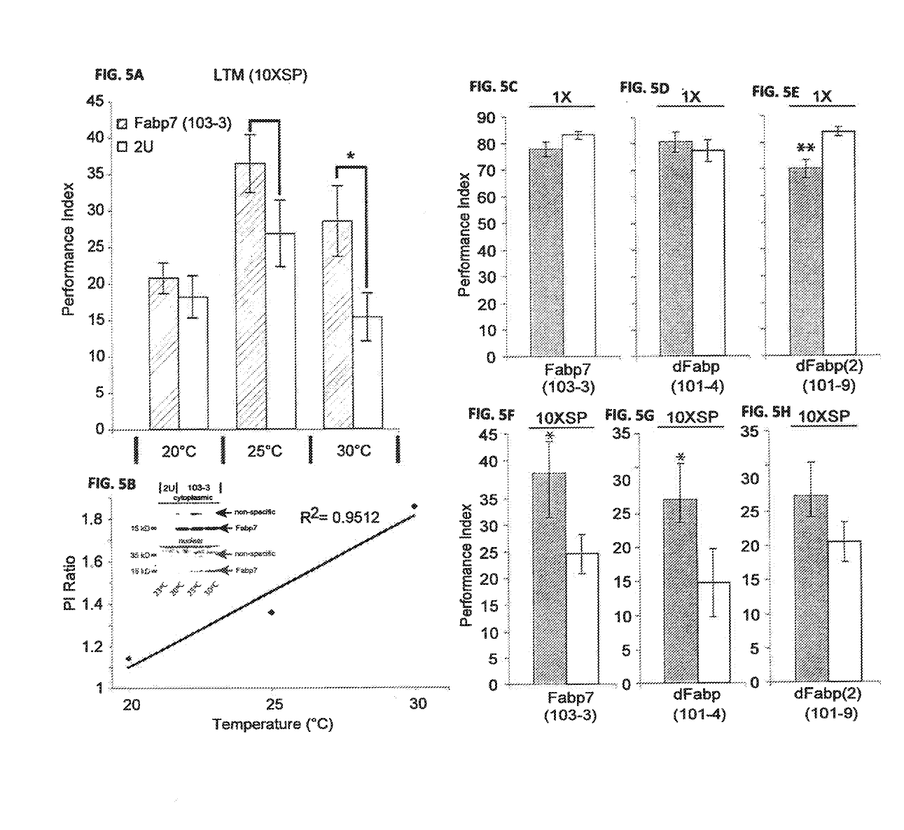

To test the effects of mild FABP7 or dFABP overexpression on memory formation, we utilized temperature shift experiments, and the results are shown in FIGS. 5A-5H.

In FIG. 5A, wild type (white bars), and FABP7 overexpressing flies (Fabp7, gray bars) were entrained on a circadian cycle at 25° C., and behaviorally trained with 10× spaced training beginning at ZT=9. After training, each genotype of flies was divided into 3 subgroups which were stored at 18, 25 or 30° C. for 7 days under light:dark cycling, at which time the 6 different groups (2 genotypes×3 conditions) were tested.

FIG. 5A shows that FABP7 overexpressing flies trained and stored at 30° C. show significant enhancement of their LTM (relative to 2U controls treated identically). Measurements are SEM. N=7-8 for each group. *p<0.05, t-test. For flies trained and stored at 25° C., there is a non-significant trend towards transgenic enhancement (p=0.06), while there is no difference for the flies stored at 20° C. FIG. 5B plots the ratio of the FABP7/2U scores as a function of temperature, showing a very nice linear relationship.

This enhancement is specific to LTM, since the Fabp7 line (103-3), and two different dFABP overexpressing lines (101-4=dFabp, and 101-6=dFabp2) do not show better learning (immediate memory after a single training trial) than their 2U controls (FIGS. 5C-5E). For this experiment, flies were raised, trained and tested at 25° C. The FABP7 or dFABP overexpressing lines also do not enhance 24-hour memory after 10 cycles of massed training, indicating that ARM is independent of dFABP levels (data not shown). In order to demonstrate the generality of the enhancement of LTM, the FABP7 overexpressing line (103-3) and two independent dFABP overexpressing lines (together with the 2U controls) were trained with 10 cycles of spaced training and stored for 7 days at 25° C. (FIGS. 5F-5H). When they were tested for 7-day memory, the FABP7 line and one of the two dFABP lines showed enhanced memory, while the other one was not significant. N=8 for all groups. * p<0.05, t-test. In order to rule out effects on retrieval, separate experiments were done where spaced trained flies were stored at 25° C. until just prior to testing, when they were shifted up in temperature. This change in temperature was actually detrimental to their subsequent performance (data not shown).

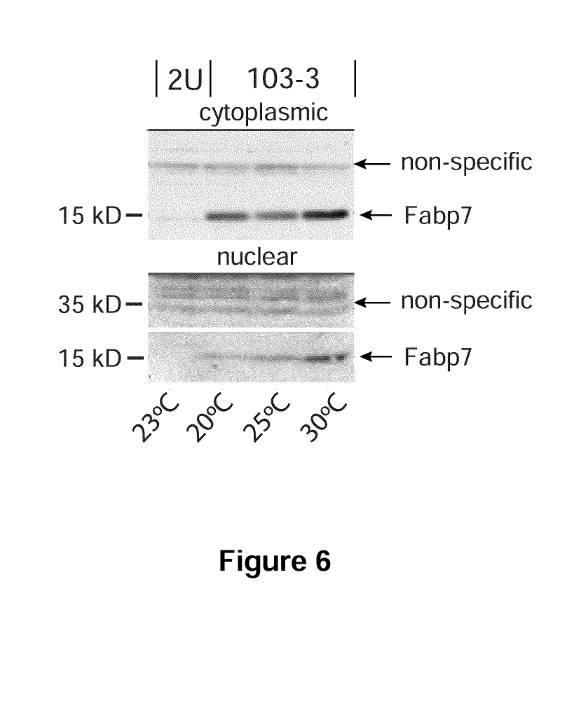

FIG. 6 shows the increase in dFABP protein as a function of temperature in both nuclear and cytoplasmic compartments from head extracts. FABP7-overexpressing flies (103-3) are shown when raised at different temperatures (20° C. to 30° C. for FABP7), and compared to wild type (2U) flies raised at 23° C. Non-specific immunoreactivity serves as loading controls.

From this data, several conclusions can be drawn. Mild over-expression of FABP7 or dFABP specifically enhances LTM because it does not enhance learning or ARM. This effect is likely to be through effects on consolidation because the flies are all trained under identical conditions and the temperature shift occurs after acquisition. Also, a shift up in temperature has a negative effect on retrieval. Therefore, FABP7 and dFABP probably affect memory consolidation.

Our data shows that moderately increasing the levels of FABP7 or dFABP alters the daytime sleep profile of the transgenic flies, and enhances memory formation. All of our manipulations involve using chronic changes in temperature to affect changes in transgene expression, thus affecting dFABP protein levels. It is well established that heat has effects on circadian cycling, sleep, and memory formation. We always included control flies (2U) in the experiments, and thus have at least one assay for non-specific effects of heat (the non-effect of heat-shock on the 2U control lines).

Therefore, the experimental approach was switched entirely to using the doxycycline (tetracycline)-inducible system to corroborate the above data, demonstrating similar effects on sleep and memory formation. Four quadrants are used: the effects of overexpressing dFABP on sleep, and on memory formation, and the effects of knocking down dFABP on sleep, and on memory formation. The doxycycline-inducible transgenes are used.

Behavioral Training Affects Sleep:

We have also shown that knocking down dFABP affects sleep. dFABP could be participating in neuronal circuits that regulate sleep, and independently, could also be affecting circuits that are involved in memory formation. In order to link these functions, it is necessary to show that dFABP is affecting a particular sleeping period, and that the affected sleeping period is impacting memory formation. One way to gather this type of data is to show that sleep deprivation following behavioral training disrupts memory formation, and that dFABP is able to overcome this negative effect. Therefore it is important to identify “windows” of time after training during which sleep deprivation affects memory formation.

We started with the simplest assumption—that flies might increase their sleep immediately following the end of training (10 cycles of spaced training). Our pilot experiment asked if there is a significant increase in post-training sleep beginning immediately after training and extending for several days. The goal is identify “windows” of increased sleep post-training, to target these windows using sleep deprivation (because it seems likely that these would be sleep times that are susceptible to deprivation), and then to ask if mild dFABP overexpression can overcome the (presumed) negative effects of deprivation on memory formation.

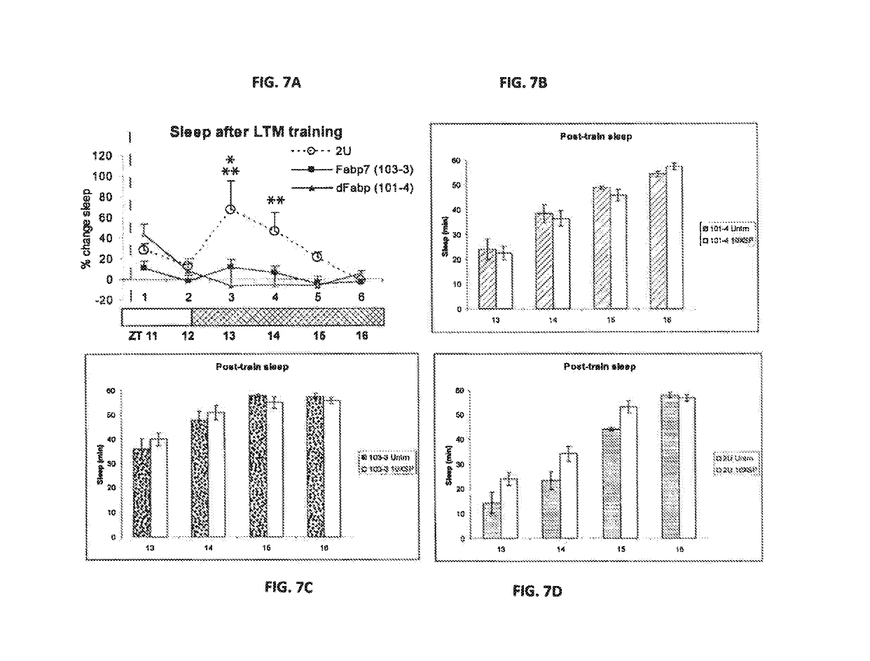

Wild-type control (2U), FABP7 (103-3), or dFABP (101-4) overexpressing flies were entrained to a circadian cycle, and given 10 cycles of spaced training, or not, beginning at ZT=7.3. The trained versus untrained flies of each genotype were then put into the sleep monitors, and their sleep patterns were measured for 1 week's time. FIG. 7A plots the percentage change in sleep (trained/untrained) for the three different genotypes that were tested: 2U (our wild type fly, dotted line), 103-3 (the FABP7 overexpressors, dark squares), and 101-4 (the dFABP overexpressors, gray triangles). The circadian cycle that was used to entrain the flies is shown below the graph. The numbers on the X-axis indicate the elapsed time since the end of 10 cycles of spaced training. For the first 6 hours after training, there is a significant increase in the amount of sleep that trained 2U flies get (2U_{trained}/2U_{handled}), while there is no increase for either of the FABP7(103-3)- or dFABP(101-4)-overexpressing lines (103-3_{trained}/103-3_{handled}, 101-4_{trained}/101-4_{handled}). This increase above baseline suggests that this might be a one “window” of sleep that is affected by behavioral training, and thus is potentially sensitive to sleep deprivation during this period.

For each 24-hour period following the end of training, we have analyzed daytime, nighttime and total daily: sleep time, average sleep bout length, average number of sleep bouts, maximum bout length, latency to sleep, and latency to awaken. There are no other significant differences over the remaining 7 days following training (data not shown). This sleep period immediately following the end of training is a likely candidate “deprivation-sensitive” window.

We expect the 103-3 and 101-4 LTM to show less sensitivity to deprivation than that of 2U. FIGS. 7B, 7C, and 7D present the quantitation of the changes on an hour-by-hour basis for the three different genotypes. It is clear that 2U flies increase their sleep (over handled controls), at least during the first 4 hours following the end of training, while neither of the transgenes does.

The FABP Transgenic Flies Have Greater Resistance to Chronic Sleep Deprivation:

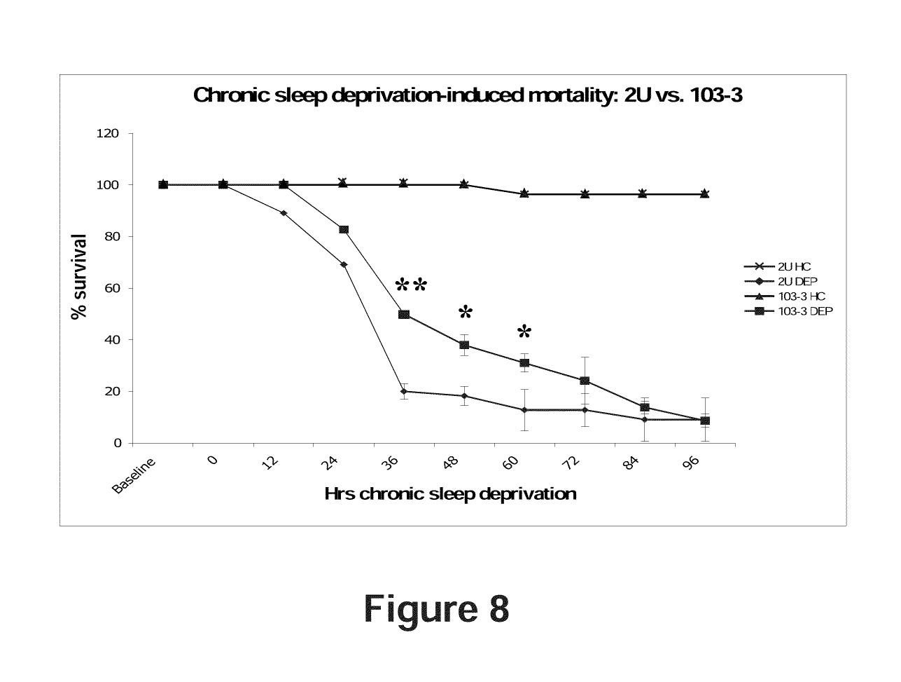

FIG. 8 shows the survival curve of a line of flies containing the mouse FABP7 gene (103-3) compared to their isogenic, wild type (2U) controls. Flies are placed in a mechanical apparatus that manually jostles the flies, preventing them from falling asleep. Lethality is measured every 6 hours and plotted as a function of time since the start of deprivation.

There is a clear difference between the survival of the flies. *p<0.05, **p<0.01 (t-test). Because the flies do not get any sleep during this assay, the conclusion is that dFABP flies are better able to deal with deprivation-induced stress, either because they got “better sleep” the night before, or because their waking stress pathways are more efficient at de-toxifying the accumulation of stress products.

The dFABP Transgenic Flies Show Faster Recovery from Anoxia

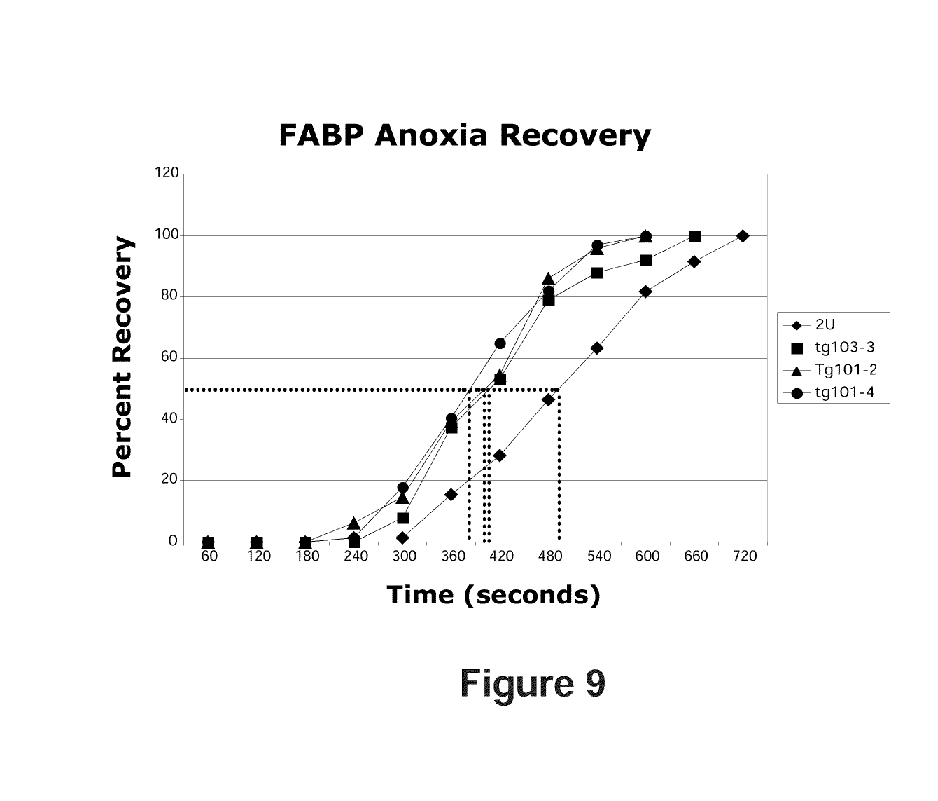

Anoxia (achieved using exposure to 100% nitrogen), is commonly used to investigate an organism's ability to de-toxify harmful products from oxidative stress. In this assay, flies were placed into a 100% nitrogen environment for 5 minutes, during which time they pass out very quickly. The flies were then removed and allowed to recover. The kinetics of recovery reflect the ability of their mitochondria and endoplasmic reticulum to handle oxidative damage. In FIG. 9, the wild type (2U) recovery curve is shown, along with the recovery curves for two independent dFABP transgenic lines (101-2 and 101-4). The fourth stock is a transgenic line carrying the mouse FABP7 gene (103-3). All three transgenic lines recovered faster than their isogenic control fly in which the transgene was inserted. The 50% recovery times are indicated with the solid lines.

dFABP Transgenic Flies are More Resistant to Hyperoxia:

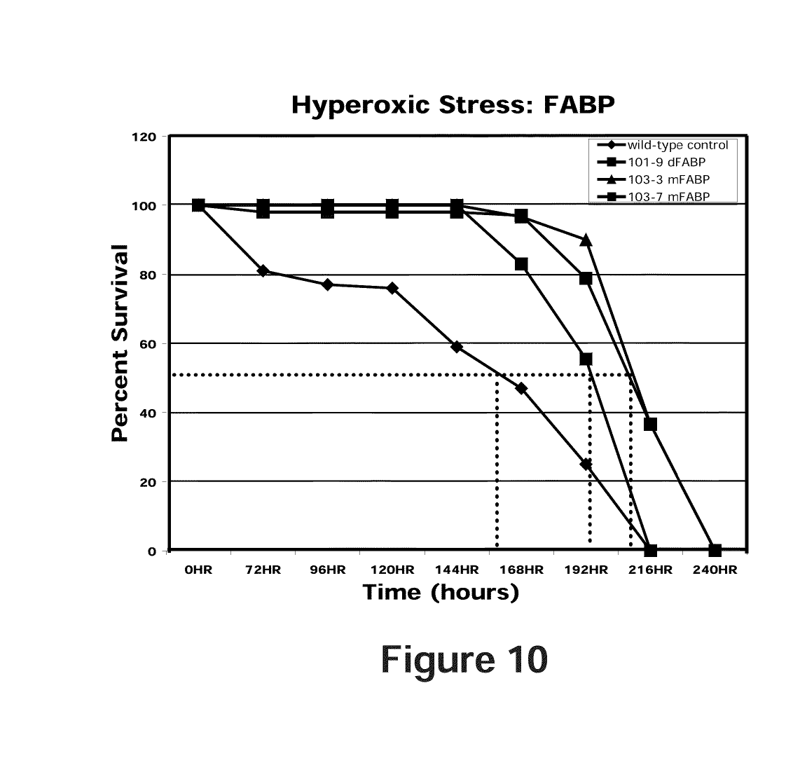

Another commonly used assay for measuring the ability of an organism's mitochondria and endoplasmic reticulum to handle oxidative damage is their survival in a hyperoxic environment. FIG. 10 shows the kinetics of death for the wild-type strain (triangles), and three different transgenic lines carrying dFABP (square) or mouse FABP7 (triangle and rectangle). Again, the resistance of the three transgenic lines is greater; their survival curves are pushed to the right.

FIGS. 8-10 show data consistent with the conclusion that transgenic flies that mildly overexpress dFABP or (mouse) FABP7 are more resistant to stress-inducing conditions, including conditions with opposite effects (anoxia vs. hyperoxia).

Collectively, these data suggest that the transgenic flies are better able to survive daytime stressors (chronic sleep deprivation, anoxia, hyperoxia), either because they detoxify faster, or had “better sleep” during the previous night.

The foregoing description and examples have been set forth merely to illustrate the invention and are not intended to be limiting. Since modifications of the disclosed embodiments incorporating the spirit and substance of the invention may occur to persons skilled in the art, the invention should be construed broadly to include all variations falling within the scope of the appended claims and equivalents thereof. Furthermore, the teachings and disclosures of all references cited herein are expressly incorporated in their entireties by reference.

What is claimed is:

1. A method for improving long-term memory, cognition, and sleep in an animal subject, the method comprising:
administering to the subject a long-term memory-improving, cognition-improving, and/or sleep-improving amount of a composition of matter that increases expression of at least one endogenoue gene that encodes at least one fatty acid binding protein (“FABP”) within the animal subject.

2. The method of claim 1 comprising administering to the subject a long-term memory-improving, cognition-improving, and/or sleep-improving amount of a composition comprising a FABP agonist in combination with a pharmaceutically suitable carrier.

3. The method according to claim 2, wherein the composition comprises a FABP7 agonist.

4. The method according to claim 2, wherein the FABP agonist is a siRNA oligonucleotide.

5. A method of testing compounds for their ability to increase expression of at least one FABP gene, the method comprising:
a) inserting into host cells an expression vector comprising a polynucleic acid sequence encoding a FABP promoter operably linked to a polynucleic acid sequence encoding a reporter gene to yield untreated transgenic host cells; and
b) exposing a portion of the untreated transgenic host cells of step (a) to at least one compound to yield treated transgenic host cells; and
c) measuring expression of the reporter gene in the treated transgenic host cells of step (b) and measuring expression of the reporter gene in the untreated transgenic host cells of step (a); and then;
d) comparing expression of the reporter gene in the treated and untreated transgenic host cells; and
e) determining if the treated transgenic host cells express more gene product from the reporter gene than the untreated transgenic host cells.

6. The method of claim 5, wherein the host cell is exposed to the at least one compound in vitro.

7. The method of claim 5, wherein the host cell is exposed to the compound being tested in vivo.

8. The method of claim 5, wherein the report gene encodes a luciferase or green fluorescent protein.

9. The method of claim 5, wherein in step (a), the host cell is a cell selected from the group consisting of bacterial cells, yeast cells, insect cells, and mammalian cells.

10. The method of claim 9, wherein the host cell is originated from a tissue selected from the group consisting of central nervous system cells.

11. The method of claim 9, wherein the host cell is unicellular.

12. The method of claim 9, wherein the host cell is part of a multicellular tissue or organism.

13. The method of claim 9, wherein the host cell is contained with a living fruit fly.

14. The method according to claim 5, wherein the FABP promoter is selected from the group consisting of a FABP3 promoter, a FABP5 promoter, a FABP7 promoter, and a dFABP promoter.
